(19) United States
(12) Patent Application Publication
(10) Pub. No.: US 2025/0277265 A1
(43) Pub. Date: Sep. 4, 2025

(54) MASS SPECTOMETRY-BASED METHOD FOR DE NOVO DIRECT SEQUENCING OF RNA MIXTURES

(71) Applicant: New York Institute of Technology, Old Westbury, NY (US)

(72) Inventor: Shenglong Zhang, Woodcliff Lake, NJ (US)

(21) Appl. No.: 18/912,030

(22) Filed: Oct. 10, 2024

Related U.S. Application Data
(60) Provisional application No. 63/589,134, filed on Oct. 10, 2023.

(51) Int. Cl. C12Q 1/6872

(52) U.S. Cl. CPC C12Q 1/6872

(57) ABSTRACT

The present disclosure provides a novel de novo RNA sequencing method which allows sequencing of each RNA and modification within a sample including sequencing of RNA modifications, while also identifying the presence and further sequencing of RNA impurities, e.g., within the therapeutic RNA sample. The method is based on the development of a novel algorithm which segregates MS data of controllable formic acid hydrolyzed RNA ladders into distinct layers based on mass, intensity and retention time allowing all RNA sequences, including their nucleotide modifications, in a mixture to be read de novo layer-by-layer.

### CROSS REFERENCE TO RELATED APPLICATIONS

This application claims the benefit of and priority to U.S. Provisional Application No. 63/589,134, filed on Oct. 10, 2023, the entire contents of which being incorporated by reference herein in its entirety.

### GOVERNMENT SUPPORT STATEMENT

This invention was made with government support under R01HG012853 awarded by the National Institutes of Health. The government has certain rights in the invention.

### REFERENCE TO ELECTRONIC SEQUENCE LISTING

The application contains a Sequence Listing which has been submitted electronically in XML format and is hereby incorporated by reference in its entirety. Said XML copy, created on May 26, 2025, is named “2637-7 US.xml” and is 31.677 bytes in size. The sequence listing contained in this .XML file is part of the specification and is hereby incorporated by reference herein in its entirety.

### TECHNICAL FIELD

The present disclosure provides a novel de novo RNA sequencing method which allows sequencing of each RNA and modification within a sample including sequencing of RNA modifications, while also identifying the presence and further sequencing of RNA impurities. e.g., within the therapeutic RNA sample. The method is based on the development of a novel algorithm which segregates MS data of controllable formic acid hydrolyzed RNA ladders into distinct layers based on mass, intensity and retention time allowing all RNA sequences, including their nucleotide modifications, in a mixture to be read de novo layer-by-layer.

### BACKGROUND

The rising prevalence of RNA-based drugs and vaccines highlights the imperative need for comprehensive detection and sequencing of all RNA species, including minor and modified variants. The presence of minor components in small chemical drugs can lead to substantial safety risks. Likewise, the failure to accurately identify co-existing RNA molecules and their modifications in therapeutic RNA reagents may result in inefficient clinical trials and misinterpreted findings. Therefore, it is essential to have the capability to comprehensively sequence mixed RNA samples, as therapeutic RNA reagents rarely achieve 100% purity, e.g., due to the limitations of current phosphoramidite chemistry in each nucleotide synthetic cycle and the subsequent purification. In fact, achieving a purity level exceeding 98.5% for RNA drugs, as required by U.S. regulatory standards for small chemical drugs, remains a substantial challenge. Inefficiencies in RNA synthesis result in samples with a variety of RNA molecules of different lengths, especially those longer than 20 nucleotides, necessitating holistic RNA sequencing techniques.

Furthermore, therapeutic RNAs often contain chemical modifications that complicate their detection and sequencing. These modifications are crucial for enhancing the stability, efficacy, and specificity of RNA vaccines and therapeutics, which are increasingly used in disease prevention and treatment¹. Numerous biopharmaceutical companies are currently developing a wide range of RNA-based therapeutics and vaccines, including miRNA and siRNA-based therapeutics, mRNA vaccines², and CRISPR-based immunotherapies³. Single guide RNA (sgRNA) in the CRISPR/Cas9 system, for instance, is essential for guiding Cas9 nuclease to recognize specific DNA sequences. sgRNA usually consists of about 100 nucleotides, and often undergoes modifications to improve stability and editing efficiency. Due to the inefficiencies in the RNA synthesis, sgRNA samples typically include various RNA molecules, each with unique sequences or modifications, demanding precise sequencing for both patient safety and regulatory compliance.

Despite wide applications, next generation sequencing (NGS)-based RNA sequencing, reliant on cDNA synthesis and either ignoring RNA modifications altogether or selectively focusing on certain RNA modifications, falls short in providing a comprehensive sequence and modification profile. Traditional mass spectrometry (MS) techniques like liquid chromatography tandem mass spectrometry (LC-MS²) promise unbiased analysis and have been used to verify therapeutic RNA sequences of interest. However, these methods typically confirm the presence of a single target RNA component⁴ and do not provide clarity on the sample's purity or identify coexisting RNA impurities^{5,6}. The spectral complexity of MS² data makes it too difficult to interpret de novo, and thus requires complementary methods (prior sequence input or knowledge) for location information. Additionally, this data complexity typically limits its application to analysis of relatively short (˜17 nt) RNA oligonucleotides. For therapeutic RNAs longer than 20 nts, these methods require additional nuclease fragmentation and subsequently meticulous LC fragment separation, further complicating the process.

Accordingly, novel RNA sequencing methods are needed for efficient analysis of RNA samples for rapid and efficient sequencing of RNAs, such as therapeutic RNAs. Such methods hold the promise to serve as a reliable quality control and regulatory standard for sequencing and verification of synthetic therapeutic RNA reagents, thereby ensuring the highest safety for patients.

### SUMMARY

The current disclosure is related to a novel MS ladder RNA sequencing method, referred to herein as the Next Generation Mass Spectrometry-based Sequencing (NGMS-Seq) method, which can be used to determine RNA sequences and identify and locate RNA modifications de novo within mixed RNA samples, without the need for prior cDNA synthesis. The method may also be used to detect and further sequence RNA impurities within an RNA sample.

Specifically, a sequencing method is provided that depends on controlled acid hydrolysis of RNA to generate well-define RNA ladders and an advanced instrument such as for example, a Vanquish Horizon UHPLC coupled with an Orbitrap Exploris 240 MS (Thermo Fisher Scientific)] to collect LC-MS data of resultant ladders to achieve complete sequencing coverage of full-length RNA. The present disclosure further provides a nested algorithm to analyze the LC-MS data of mixed RNA samples, allowing de novo sequencing of desired RNAs, such as therapeutic RNAs, and detection of unwanted RNA impurities within the sample. Additionally, mathematical foundations and reaction kinetics of RNA acid hydrolysis have been established and provided for in silico simulating controlled acid hydrolysis of RNA and for quantifying diverse hydrolyzed RNA fragments. Such fragments include desired ladder fragments that retain either the original 5′ end or the 3′ end of the parental RNA molecule and are critical for MS sequencing, internal fragments that do not have either the original 5′ end or the 3′ end but complicate the data analysis, and intact RNA molecules that remain unhydrolyzed.

In an embodiment, the present disclosure provides a system for RNA sequencing comprising the following steps: (i) RNA sample preparation including acid hydrolysis, (ii) LC-MS measurement of RNAs intact before and after acid hydrolysis to collect data for MS sequencing and analysis, and (iii) data processing for de novo sequencing of RNA and their modifications through use of the disclosed layer-by-layer nested sequencing algorithm and, optionally, a sequencing scoring system thereby leading to sequence alignment and assembly.

The steps of the method and Nested Algorithm are based both on empirical observations and theoretical considerations of RNA hydrolysis patterns. Key characteristics of the designed method include: (i) the occurrence of diverse types of fragments quantified through the average ratio of hydrolysis per nucleotide, defined as rₐᵥ, which is critical for sequencing and inherently reliant on hydrolysis time. (ii) the MS intensity ratio between internal fragments and desired ladder fragments which approximates the rₐᵥ value, which can be controlled via well-designed experimental conditions; and (iii) the MS intensity order of desired ladder fragments from different parent RNAs which typically aligns with the abundance order of the original RNAs in the sample.

This presently disclosed method represents a significant advancement as a new dimension has been added to aid in data separation 3D mass/retention time/intensity plots. Using optimal RNA hydrolysis and LC-MS conditions through two control runs to attain the rₐᵥ value, and in silico simulation based on mathematical foundations and reaction kinetics of RNA acid hydrolysis, the fraction of desired ladder fragments are increased; whereas, undesired internal fragments are minimized. The provided Nested Algorithm, takes advantage of the hydrolysis pattern of RNAs in the sample to derive RNA sequences based on their abundance order in the original RNA sample and the resulting ladder fragments maintaining the same relative intensity. i.e., the most abundant RNA generated the most abundant ladder fragments in the sample. Using this 3D approach significantly improves the accuracy and efficiency in sequencing multiple RNAs within a complex mixture.

The present disclosure provides a kit for use in generating the sequence of one or more RNA molecules, detecting the presence, identity, location, and quantity of RNA nucleotide modifications, and/or possible impurities, on said one or more RNA molecules, said kit comprising one or more components for performance of a method comprising one or more of the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) processing of data through use of the disclosed nested algorithm and, optionally, the disclosed sequencing scoring system, thereby generating the sequence of one or more RNA molecules, detecting the presence, identity, location, and quantity of RNA nucleotide modifications and/or the presence of possible impurities.

The present disclosure provides a computer-implemented method for determining an order of nucleotides and/or modifications of an intact RNA or a controlled enzymatically cleaved RNA molecule, wherein the method includes: receiving/exporting liquid chromatography-mass-spectrometry (LC-MS) data of an RNA sample, the LC-MS data including but not limited to a mass (e.g., m/z, monoisotopic mass, average mass), charge states, retention time (t_{R}), Hight, width, volume, relative abundance, and quality score (QS); filtering the LC-MS data based on mass, the filtering including removing masses smaller than a predetermined size; analyzing the filtered LC-MS data, to determine a plurality of RNA sequences, and processing the LC-MS data through use of the disclosed nested algorithm and, optionally, the disclosed sequencing scoring system.

In an embodiment, provided is a non-transitory computer-readable medium storing instructions that, when executed by a processor, cause the processor to perform a method for generating the sequence of one or more RNA molecules and detecting the presence, identity, location, and quantity of RNA nucleotide modifications on said one or more RNA molecules, the method comprising the steps of (i) identifying a specific chemical moiety associated with the RNA thereby imparting an identifiable property on the RNA (ii) controlled fragmentation of the RNA to form 5′ and 3′ MS ladder fragments; (iii) mass measurement of resultant degraded RNA samples containing RNAs and their degraded fragments; and (iv) processing the LC-MS data using the disclosed nested algorithm and, optionally the disclosed sequencing scoring system, provided herein.

Accordingly, provided is a method for generating the sequence of one or more RNA molecules and detecting the presence, identity, location, and quantity of RNA nucleotide modifications on said one or more RNA molecules, allowing sequencing of each RNA and modification in a given RNA sample, said method RNA comprising (i) controlled fragmentation of the RNA sample, (ii) LC-MS measurement of RNAs intact before and after acid hydrolysis to collect data for MS sequencing and analysis, and (iii) data processing for de novo sequencing of RNA and their modifications through use of a layer-by-laver nested sequencing algorithm.

Further, a method is provided for detecting and further sequencing of impurities that co-existing with the target sequence in a RNA sample, allowing sequencing of each RNA and modification in a given RNA sample, said method RNA comprising (i) controlled fragmentation of the RNA sample. (ii) LC-MS measurement of RNAs intact before and after acid hydrolysis to collect data for MS sequencing and analysis, and (iii) data processing for de novo sequencing of RNA and their modifications through use of a layer-by-layer nested sequencing algorithm. Said method provides the relative abundance of the impurities. In said method the algorithm segregates data points of consecutive ladder fragments from the same parent RNA based on the original RNA abundance order into a 3D-mass-intensity-retention time layer. Additionally, within each layer, a short RNA sequence is read by base-calling each nucleotide from mass differences between consecutive ladder fragments thereby efficiently handling MS data separation and separate different RNA sequences on silica, eliminating the need for complex physical sample separation steps.

Further, a Nested Algorithm is provided comprising the steps of initiating the 1^{st} layer by identifying the data point (data point A) with a mass lower than the parent RNA and which has the highest signal intensity (i.e., the most intense potential ladder fragment) and data point A is likely associated with a ladder fragment from the most abundant RNA in the sample; (ii) starting from point A, base calling which identifies the subsequent data point (data point B) with an added mass equivalent to one nucleotide wherein point B likely corresponds to a consecutive ladder fragment from the same RNA and recording as an additional nucleotide in the RNA sequence; once data point B is identified, continuation of the process iteratively to determine subsequent data points of RNA ladder fragments.

In such a Nested Algorithm searching for the next data point follows one of more of the following criteria: 1) alignment of mass differences with the searching library, encompassing four canonical nucleotides and RNA modifications: 2) fitting appropriate retention time (t_{R}) differences into a 2D t_{R} versus mass sigmoidal curve; 3) selecting the match with highest intensity in cases of multiple matches; and 4) ensuring intensities within the same order of magnitude for consecutive ladder fragments.

Further details and aspects of exemplary embodiments of the disclosure are described in more detail below with reference to the appended figures. Any of the above aspects and embodiments of the disclosure may be combined without departing from the scope of the disclosure.

### BRIEF DESCRIPTION OF THE DRAWINGS

This patent or application file contains at least one drawing executed in color. Copies of this patent or patent application publication with color drawing(s) will be provided by the Office upon request and payment of the necessary fee. Various embodiments of methods are described herein with reference to the drawings wherein:

FIG. 1A-C. Sequencing workflow using the nested algorithm.

FIG. 1A. Two control runs provide the information for the exact number of RNAs in the sample and their relative abundances and offer insights into optimal experimental conditions and LC-MS configurations to be employed in the subsequent analytical run; FIG. 1B. Main steps of the proposed layer-by-layer nested sequencing algorithm. The starting point of each layer is the strongest data point (1^{st} potential ladder fragment of this layer) among the current data pool, followed by searching for the next and the previous ladder fragments and base calling. *The desired next and previous ladder fragments should satisfy the four requirements on (1) mass difference between consecutive ladder fragments: (2) retention time differences; and (3) (4) two aspects for intensity requirements (SEQ ID NO:1; SEQ ID NO: 35); FIG. 1C. The layer-by-layer ladder fragment searching process will continue until all strong data points have been examined. The sequence segments read out in different layers will be aligned and assembled to enhance the final output.

FIG. 2A-E. RNA sequencing by controlled acid hydrolysis/LC-MS.

FIG. 2A. A synthetic 20-nt RNA with a hydroxyl group (orange triangle) at the 3′ end. In the green box, the formulas for calculating the site-specific phosphodiester hydrolysis rate and the hydrolysis fraction r₁, respectively, were given. FIG. 2B. A general reaction scheme for acid-catalyzed hydrolysis of ribonucleotide phosphodiester bond. Under acidic conditions, the phosphodiester hydrolysis generates a 3′ ladder fragment and a 5′ ladder fragment. FIG. 2C. A demonstration of 5′ and 3′ ladder fragments (retaining the original 5′ and 3′ end of the RNA), and internal fragments after controlled acid-catalyzed hydrolysis of RNA (20 nts). The intact RNA (unhydrolyzed), 5′ ladder fragments. 3′ ladder fragments, and internal fragments (RNA is cut more than once) are colored green, blue, red, and grey, respectively. Purple circles: phosphate groups; orange triangles: hydroxyl groups. Black diamonds: “Head” groups and orange arrows: “Tail” groups at the 5′ and 3′ ends of the original RNAs. FIG. 2D. Formulas for calculating the monoisotopic mass values of four different types of fragments after acid hydrolysis. Supposing the length of the original RNA is m nucleotides, the fragments are of length k after cleaving the original RNA molecule at position k (the k^{th} phosphodiester from 5′ end). M_{H}: the mass of the “Head” group; MT: the mass of the “Tail” group. Also shown are the ladder fragments generated by controlled acid hydrolysis and their mass difference is used for base calling FIG. 2E. Formulas for calculating the proportion of four different types of fragments after acid hydrolysis (probability formulas). Supposing the length of the original RNA is m, the fragments are of length k after cleaving the original RNA molecule at position k (the k^{th} phosphodiester from 5′ end).

FIG. 3A-B. NGMS-Seq 3D separation of a 100 nt synthetic RNA and a mixture of three 20 nt RNAs. FIG. 3A. The average hydrolysis rate rₐᵥ can be used to approximate the occurrence of different ladder fragments. The intensity ratio between consecutive 3′ ladder fragments (3 nts-71 nts) after acid hydrolysis of a 100 nt synthetic RNA, has a mean value of 1. FIG. 3B. Left: relative intensity of original RNA in the control sample (three 20 nt synthetic RNAs); right: relative intensity of the 10 nt ladder and internal fragments from three different 20 nt synthetic RNAs after controlled acid hydrolysis. The intensity ratio (i.e., amount ratio) of different RNAs roughly remains the same (˜9:3:1) before and after controlled acid hydrolysis.

FIG. 4A-C. NGMS-Seq result for a three 20 nt RNA mixture. FIG. 4A. The monoisotopic mass and the relative intensity of the control mixture of three 20 nt synthetic RNAs (RNA-A, RNA-B. and RNA-C). Et₃N: Triethylamine (N(CH₂CH₃)₃), existing in the solvent for running LC-MS. Relative intensity, the intensity ratio between an RNA and the most abundant RNA in a sample, which reflects the RNA's relative amount compared to the most abundant RNA. FIG. 4B. The sequence coverage and depth of the three RNAs (RNA-A. RNA-B. and RNA-C) in the mixture using the results of 15-layers. (SEQ ID NO: 2-4) FIG. 4C. The monoisotopic mass (Da)-retention time (min) 2D plot of identified 5′ and 3′ ladders of RNA-A, RNA-B, and RNA-C. The color bar indicates the log₁₀ (Intensity) for each ladder fragment. The read out sequences based on each set of ladder fragments are annotated (SEQ ID NO: 5-10).

FIG. 5A-E. FIG. 5A. sgRNA2 (100 nt) sequence and sequencing coverage by its original form of 5′ and 3′ ladders. (SEQ ID NO. 11-13). Green: 5′ ladder coverage; Orange: 3′ ladder coverage. FIG. 5B. Position and stoichiometry of non-canonical nucleotides in sgRNA2. FIG. 5C. The monoisotopic mass (theoretical and observed mass) and (relative intensity of detected RNA molecules >3%) in the commercially purchased sgRNA2 sample (acid hydrolysis sample). FIG. 5D. site-specific intensity of 5′ (upper) and 3′ (lower) ladder fragments, colored by log₁₀ (Intensity). FIG. 5E the retention time (min)-monoisotopic mass (Da) plot of sgRNA2 5′ and 3′ ladders, colored by log₁₀ (Intensity). “x” cross shape: 5′ ladder fragments; Solid triangles: 3′ ladder fragments; star shape: intact sgRNA2.

FIG. 6. Sequencing coverage obtained using different sgRNA mixtures with varying initial sample ratios. Blue: sgRNA1 percent in the sample. Orange sgRNA2 percent in the sample. The coverage combines the results from the 5′ ladder, dehydrated 5′ ladder f, 5′ ladder sodium and potassium adducts, the 3′ ladder, and the 3′ sodium and potassium adducts.

FIG. 7. Nested algorithm identifies impurities in the sgRNA1 sample. Several contaminants found in the sgRNA1 sample. (SEQ ID NO: 14-26)

FIG. 8. Nested algorithm flowchart. The flowchart shows the logic behind the nested algorithm. Three main sections: 1) Data cleaning (noise reduction); 2) Identify ladder fragments and base-calling based on the four criteria on mass, retention time, and intensity: 3) Result refinement including sequence alignment and assembly to output the final sequencing results.

FIG. 9. Picture of 1D, 2D and 3D data separation. 3D data separation strategy is the most practical and effective approach in separating the ladder fragments from different RNAs. The 1D data separation involves only the MS signal of mass and the 2D data separation incorporates mass and retention time. Compared to 1D and 2D data separation strategies, the 3D data separation is the most practical method in grouping and separating ladder fragments of RNAs from a mixture.

FIG. 10. Parallel sequences and Gap between consecutive sequences used in sequence assembling and verification step. (1) Parallel sequences left and (2) “Gap” between consecutive sequences used in sequence assembling and verification step.

FIG. 11. Base calling pool size is greatly reduced when considering intensity during sequencing. The 100 nt synthetic RNA acid-hydrolysis data was used.

FIG. 12A-C. Intensity of Different Ladder Fragments from Three 20 nt Synthetic RNAs after Acid Hydrolysis. FIG. 12A. Intensity of ladder fragments of different lengths and average intensity of internal fragments with the same length after controlled acid hydrolysis. Horizontal colored bands indicate the respective average intensity values and 95% confidence interval. Ladder and internal fragments are from the mixture of three 20 nt synthetic RNAs (RNA-A. RNA-B. and RNA-C with an original intensity ratio of 9:3.008:0.604 in control sample); FIG. 12B, the actual maximum intensity of each form of ladder and internal fragments after acid hydrolysis in the mixture of three 20 nt synthetic RNA (RNA-A, RNA-B, and RNA-C). The ladder fragments exist in different forms, and the stronger forms include the original 3′ and 5′ ladders, 3′ and 5′ ladders with potassium adduct (K⁺), 3′ and 5′ ladders with sodium adduct (Na⁺), and dehydrated 5′ ladders (cyclic 2′,3′-phosphate intermediate form). The red horizontal line represents the intensity value of about 10% of the strongest 3′ ladder fragment of RNA-A. FIG. 12C. Example sequence and relative intensity outputs from three layers for 3′ ladder fragments of RNA-A, RNA-B. and RNA-C (obtained from Layer 1, 3, and 9, respectively). The bar plots show the relative intensity profile of ladder fragments identified in each layer (relative intensity of ladder fragments in each layer=ladder fragment intensity/the intensity of the strongest ladder fragment or the 1st ladder fragment obtained in this layer). (SEQ ID NO:27-29)

FIG. 13A-F. The sequencing coverage generally increases as the sgRNA sample ratio increases in the mixtures. FIG. 13A-F. Sequencing coverage obtained using different sgRNA mixtures with varying initial sample ratios; f: Sequencing coverage—sgRNA2 sample percentage. The sequencing coverage is obtained from 5′ and 3′ ladders of sgRNA1 and sgRNA2, respectively. Blue: coverage of 5′ ladder of sgRNA1; red: coverage of 3′ ladder of sgRNA1; green: coverage of 5′ ladder of sgRNA2; orange: coverage of 3′ ladder of sgRNA2. The coverage from 5′ ladder combines the coverage from 5′ ladder original form, dehydrated form, sodium and potassium adduct form, and the coverage from 3′ ladder combines the coverage from 3′ ladder original form, sodium and potassium adduct form.

FIG. 14A-D. Sequencing Result of sgRNA1. FIG. 14A. sgRNA1 (100 nt) sequence and sequencing coverage by its original form of 5′ and 3′ ladders Blue: 5′ ladder coverage; Red: 3′ ladder coverage. (SEQ ID NO: 30-32) FIG. 14B. The monoisotopic mass (theoretical and observed mass) and RI of detected RNA molecules (RI>3%) in the commercially purchased sgRNA1 sample (acid hydrolysis sample). FIG. 14C. site-specific intensity of 5′ (upper) and 3′ (lower) ladder fragments, colored by log 10 (Intensity). (SEQ ID NO: 33-34) FIG. 14D, the Retention Time (min)-Monoisotopic Mass (Da) plot of sgRNA1 5′ and 3′ ladders, colored by log 10 (Intensity). Solid Circles: 5′ ladder fragments; Diamond Shapes: 3′ ladder fragments; Star Shape: intact sgRNA1

FIG. 15A-F. Simulated percentage of fragments generated after controlled acid hydrolysis. The 3-D graphs above show the calculated percentage of all fragments generated (vs. the original amount of the hydrolyzed RNA) after controlled acid hydrolysis. A set of 20 site-specific fractions of hydrolyzed ri (i=1-20) were randomly generated with the specified rₐᵥ and relative standard deviation (A: Rav=1.0%, rsd=29%; B: Rav=2.0%, rsd=30%; C: Rav=5.0%, rsd=24%; D: Rav=10%, rsd=27%; E: Rav=20%, rsd=27%; F: Rav=50%, rsd=28%). X-axis: positions from 5′- to 3′-ends labelled as 1 to 20. Y-axis: positions from 5′- to 3′-ends labelled as 1 to 20. Z-axis: percentage of the corresponding fragments.

### DETAILED DESCRIPTION

Although the present disclosure will be described in terms of specific embodiments, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The current disclosure is related to an RNA sequencing method, referred to herein as Next Generation Mass Spectrometry-based Sequencing (NGMS-Seq), which can be used to determine the nucleotide sequence of RNA molecules with single nucleotide resolution as well as detection of the presence of any nucleotide modifications and impurities that an RNA molecule/sample may carry. The RNA to be sequenced may be a purified RNA sample of limited diversity, as well as samples of RNA containing complex mixtures of RNA, such as RNA derived from a biological sample.

As used herein, ribonucleic acid (RNA) refers to oligoribonucleotides or polyribonucleotides as well as any analogs of RNA, for example, made from nucleotide analogs. The RNA will typically have a base moiety of adenine (A), guanine (G), cytosine (C) and uracil (U), a sugar moiety of a ribose and a phosphate moiety of phosphate bonds. RNA molecules include both natural RNA and artificial RNA analogs. The RNA can be synthetic or can be isolated from a particular biological sample using any number of procedures which are well known in the art, wherein the particular chosen procedure is appropriate for the particular biological sample. RNA samples include for example, coding RNA and non-coding RNA such as mRNA. IRNA, IRNA, antisense-RNA, and siRNA, to name a few. No limitations are imposed on the base length of RNA. The MLC-Seq sequencing methods disclosed herein enable the sequencing of not only purified RNA samples, but also more complicated RNA samples containing mixtures of different RNAs.

In a specific embodiment, the structure of synthetic oligoribonucleotides of therapeutic value, as well as the presence of impurities, can be determined using the sequencing methods disclosed herein. Such methods will be of special valuable to those engaged in research, manufacture, and quality control of RNA-based therapeutics, as well as the regulatory entities. Incorporation of structural modifications into synthetic oligoribonucleotides has been a proven strategy for improving the polymer's physical properties and pharmacokinetic parameters. However, the characterization and the structure elucidation of synthetic and highly modified oligonucleotides remains a significant hurdle.

The current disclosure is related to a novel MS ladder RNA sequencing method, referred to herein as the Next Generation Mass Spectrometry-based Sequencing (NGMS-Seq) method, which can be used to determine RNA sequences and modifications de novo within mixed RNA samples, without the need for prior cDNA synthesis. The method may also be used to detect impurities within an RNA sample.

Specifically, a sequencing method is provide that depends on controlled acid hydrolysis of RNA to generate well-define RNA ladders and an advanced instrument to collect LC-MS data of resultant ladders to achieve complete sequencing coverage of full-length RNA (e.g., 100 nt sgRNA). The present disclosure further provides a nested algorithm to analyze the LC-MS data of mixed RNA samples, allowing de novo sequencing of desired therapeutic RNAs and detection of unwanted RNA impurities. Additionally, mathematical foundations and reaction kinetics of RNA acid hydrolysis are provided for in silico simulating controlled acid hydrolysis of RNA and for quantifying diverse hydrolyzed RNA fragments, including desired ladder fragments that retain either the original 5′ end or the 3′ end of the parental RNA molecule and are critical for MS sequencing, internal fragments that do not have either the original 5′ end or the 3′ end but complicate the data analysis, and intact RNA molecules that remain unhydrolyzed.

In an embodiment, the present disclosure provides a system for RNA sequencing comprising the following steps: (i) RNA sample preparation including controlled acid hydrolysis of an RNA sample, (ii) LC-MS measurement of RNAs intact before and after acid hydrolysis to collect data for MS sequencing and analysis, and (iii) data analysis for de novo sequencing of RNA and their modifications using the disclosed laver-by-layer Nested Sequencing Algorithm, and optionally the disclosed Sequencing Scoring System, followed by sequence alignment and assembly.

The steps of the method and nested algorithm are based both on empirical observations and theoretical considerations of RNA hydrolysis patterns. Key characteristics of the designed method include: (i) the occurrence of diverse types of fragments quantified through the average ratio of hydrolysis per nucleotide, defined as ra», which is critical for sequencing and inherently reliant on hydrolysis time. (ii) the MS intensity ratio between internal fragments and desired ladder fragments which approximates the rₐᵥ value, which can be controlled via well-designed experimental conditions; and (iii) the MS intensity order of desired ladder fragments from different parent RNAs typically aligns with the abundance order of the original RNAs in the sample.

This provided method represents a significant advancement as a new dimension has been added to aid in data separation 3D mass/retention time/intensity plots. Using optimal RNA hydrolysis and LC-MS conditions through two control runs to attain the fav value, and in silico simulation based on mathematical foundations and reaction kinetics of RNA acid hydrolysis, the fraction of desired ladder fragments are increased; whereas, undesired internal fragments are minimized. The provided Nested Algorithm, capitalizes on the hydrolysis pattern of RNAs in the sample to derive RNA sequences based on their abundance order in the original RNA sample and the resulting ladder fragments maintained the same relative intensity. i.e., the most abundant RNA generated the most abundant ladder fragments in the sample. Using this 3D approach significantly improves the accuracy and efficiency in sequencing multiple RNAs within a complex mixture.

The present disclosure provides a system for RNA sequencing comprising the following steps: (i) RNA sample preparation which includes controlled acid hydrolysis of the RNA sample, (ii) LC-MS measurement of RNAs intact before and after acid hydrolysis to collect data for MS sequencing and analysis, and (iii) data analysis for de novo sequencing of RNA and their modifications using a provided layer-by-layer nested sequencing algorithm, followed by sequence alignment and assembly. As a step in the presently provided sequencing method the RNA sample is subjected to controlled acid-based fragmentation to provide well-defined RNA ladders.

In an embodiment, chemical cleavage is accomplished through use of formic acid. Formic acid degradation is preferred because its boiling point is approximately 100° C. like water and the formic acid can be easily remove it e.g., by lyophilizer or speedvac. Such cleavage is designed to cleave the RNA molecule at its 5′-ribose positions throughout the molecule.

In addition to formic acid degradation, alkaline degradation may also be used. For example, the following alkaline buffers may be used to degrade the RNA sample: 1× Alkaline Hydrolysis Buffer (e.g., 50 mM Sodium Carbonate [NaHCO₃/Na₂CO₃] pH 9.2, 1 mM EDTA; or the Alkaline Hydrolysis Buffer supplied with Ambion's RNA Grade Ribonucleases), or ammonia aqueous solution (NH₄OH, 25%-32%). In addition to chemical cleavage. RNAs may be subjected to enzymatic degradation. Enzymes that may be used to degrade the RNA include for example, Crotalus phosphodiesterase I, bovine spleen phosphodiesterse II and XRN-1 exoribonucease. Such RNA degradation treatment is carried out under conditions where a desired single cleavage event occurs on the RNA molecule resulting in a pool of differently sized RNA fragments resulting in a complete ladder.

Once RNA fragment pools are formed, the RNA fragments, as well as intact RNA, can be analyzed by any of a variety of means including liquid chromatography coupled with mass spectrometry, or gas chromatography coupled with mass spectrometry, or ion-mobility spectrometry coupled with mass spectrometry, or capillary electrophoresis coupled with mass spectrometry, or other methods known in the art. Mass spectrometer formats include continuous or electrospray ionization (ESI) and related methods or other mass spectrometer that can detect RNA fragments like MALDI-MS, Orbitrap Exploris MS Mode: 120, 240 480. HPLC-MS measurements can be performed using high resolution time-of-flight or Orbitrap mass spectrometers that have a mass accuracy of less than 5 ppm.

LC-MS data is then converted into RNA ladder sequence information. The unique mass tag of each canonical ribonucleotide and its possibly associated modifications on the RNA molecule, allows one to not only determine the primary nucleotide sequence of the RNA but also to determine the presence, type and location of RNA modifications. When an RNA is not 100%, each of the RNA ladder fragments carries stoichiometry information, which allows stoichiometric quantification of each nucleotide modification site-specifically.

Mass adducts obtained from the deconvoluted data will be used to generate/confirm sequence generated by native ladder fragments (no adducts) using both mass, intensity and retention time data. The retention time-coupled mass data for the fragments is analyzed to determine which data points are “valid” and to be used for subsequent sequence determination and which data points are to be filtered out. After data reduction step, the mass difference (m) between two adjacent RNA fragments [m=m (i)−m(i−1), 1<j<n, n=RNA length], where m(i) is the mass of any ladder fragment and m(i−1) is the preceding lower mass ladder fragment, and match such mass differences with the exact masses of known nucleotide fragments to correlate the derived RNA sequencing information based on mass differences to determine the RNA sequence and its modification. As long as the structural modification on an RNA nucleoside is mass-altering, the disclosed sequencing method will permit identification of the RNA sequence and its modification to be identified. The mass of all the known modified ribonucleosides can be conveniently retrieved from known RNA modification databases.

The mathematical foundation and kinetics of RNA acid hydrolysis of a phosphor-ester bond (P—O bond) for nested algorithm-based NGMS-seq of RNA mixtures is as follows. For an RNA strand with m nucleotides, there are m−1 cleavable phosphodiester bonds (P—O). The acid hydrolysis of a P—O bond is a multi-step reaction. Since the protonation of the phosphate is the rate-determining step¹⁰. P—O hydrolysis follows the second order rate law and the site-specific rate constant for the P—O bond between nucleotide i and i+1 is kᵢ.

$$rate = {{k}_{i} [ P - O ]}_{i} [ {H}_{3} {O}^{+} ] \qquad Equation ( 1 )$$

Because the pH of the acid-hydrolysis is ˜2 and the concentration of RNA molecules is in the μM level, the concentration of [H₃O⁺] is relatively unchanged before and after acid treatment. Thus, the hydrolysis follows the first order rate law at each reaction site:

$${rate}_{i} = {{k}_{i}}^{′} [ P - O ] , where {{k}_{i}}^{′} = {k}_{i} [ {H}_{3} {O}^{+} ] \qquad Equation ( 2 )$$

Using the first order integrated rate law:

$$ln ( \frac{{[ P - O ]}_{it}}{{[ P - O ]}_{i 0}} ) = - {k}_{i}^{′} t \qquad Equation ( 3 )$$

where [P—O]ᵢₜ and | P—O]ᵢ₀ are the concentrations of [P—O]ᵢ at time (t) and initial time (0), and t is the hydrolysis time. The ratio [P—O]ᵢₜ/[P—O]ᵢ₀ is the fraction of [P—O] at that specific site i that remains unhydrolyzed. Define rᵢ as the fraction of hydrolyzed P—O at i^{th} site (between nucleotide i and i+1) and combining Arrhenius Equation,

$${k}_{i} = {A}_{i} {e}^{- \frac{{E}_{ai}}{RT}} ,$$

rᵢ can be expressed as:

$${r}_{i} = 1 - \frac{{[ P - O ]}_{it}}{{[ P - O ]}_{i 0}} = 1 - {e}^{- {k}_{i}^{′} t} = 1 - {e}^{- {k}_{i} [ {H}_{3} {O}^{+} ] t} = 1 - {e}^{- {A}_{i} {e}^{( - \frac{{E}_{ai}}{RT} )} [ {H}_{3} {O}^{+} ] t} \qquad Equation ( 4 )$$

Equation (4) thus reveals that the magnitude of phosphodiester bond hydrolysis at each site can be determined by experimental conditions including: (1) reaction temperature. (2) formic acid concentration, and (3) hydrolysis time.

The following may be used for determination of rₐᵥ (average ratio of hydrolysis per nucleotide). While site-specific fraction of hydrolysis rᵢ values are difficult to determine for all phosphodiester bond, their average value rₐᵥ is readily determined from the intensity ratio of the parent RNA before and after acid-hydrolysis:

$$\frac{MS intensity of remaining parent RNA after hydrolysi}{MS intensity of original parent RNA} = \frac{{{C}_{0} ( 1 - {r}_{av} )}^{m - 1}}{{C}_{0}} = {( 1 - {r}_{av} )}^{m - 1} \qquad Equation ( 5 )$$

$$Then {r}_{av} = 1 - \sqrt[m - 1]{\frac{MS intensity of remaining parent RNA after hydrolysis}{MS intensity of original parent RNA}}$$

It has been found that the values of rₐᵥ for different RNAs are very close under identical LC-MS conditions. As described below the rₐᵥ can be calculated using the intact RNA intensity ratio obtained from the two control runs.

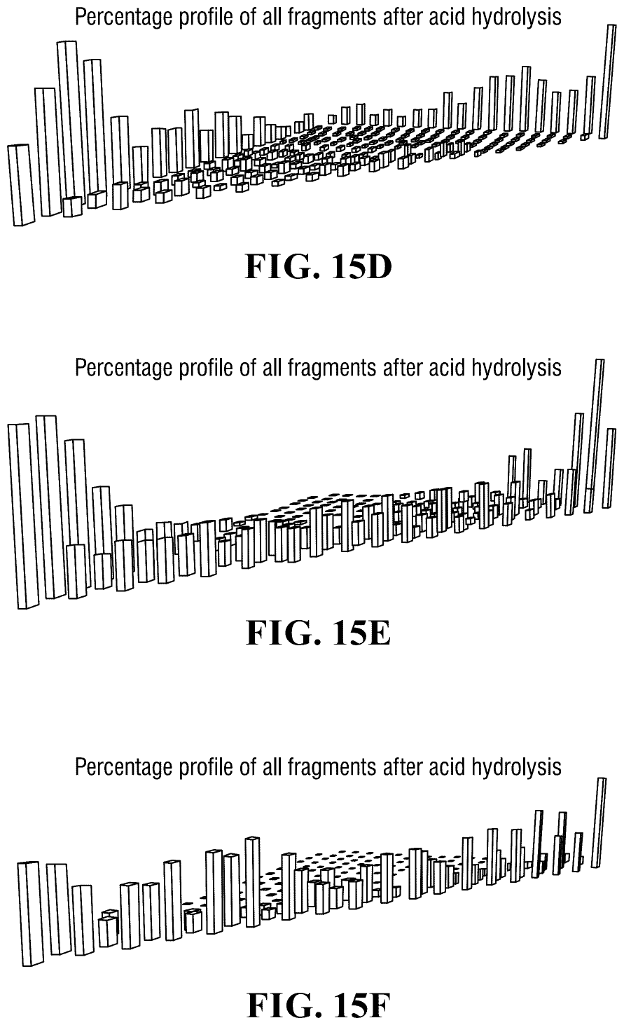

In one aspect, determination of Optimal Hydrolysis Time may be performed as follows. With the preliminary data from the two control runs and in silico simulation supported by RNA hydrolysis kinetics, the acid hydrolysis conditions are adjusted to maximize the generation of desired RNA ladder fragments while minimizing undesired internal fragments. The optimal hydrolysis time is determined from information provided in the two control runs. The length of an original RNA molecule is approximated by dividing its monoisotopic mass by 320 Da. The average hydrolysis ratio per site, rₐᵥ, is calculated using Equation (5) The theoretical optimal rₐᵥ for a ladder fragment with length k is determined as 1/k. Moreover, the value of rₐᵥ is directly proportional to hydrolysis time, and a small rₐᵥ (<0.05) allows for a proportional calculation of the theoretical optimal hydrolysis time (FIG. 15). The extensive LC-MS measurements of RNAs revealed that rₐᵥ is approximately 0.004 per minute at 40° C. under the illustrated acid-hydrolysis condition.

In one aspect, it has been found that the MS intensity order of fragments from different RNAs generally follows the abundance order of the original RNAs in the sample. The first order hydrolysis kinetics for RNAs' P—O bonds (Equation (2)) leads to the independent approximation of the probability of forming fragments based on the initial concentration of original RNAs. Therefore, all original RNAs in the sample undergo hydrolysis to a similar extent, and the intensity order of fragments with the same length from different RNAs typically aligns with the abundance order of their original RNAs.

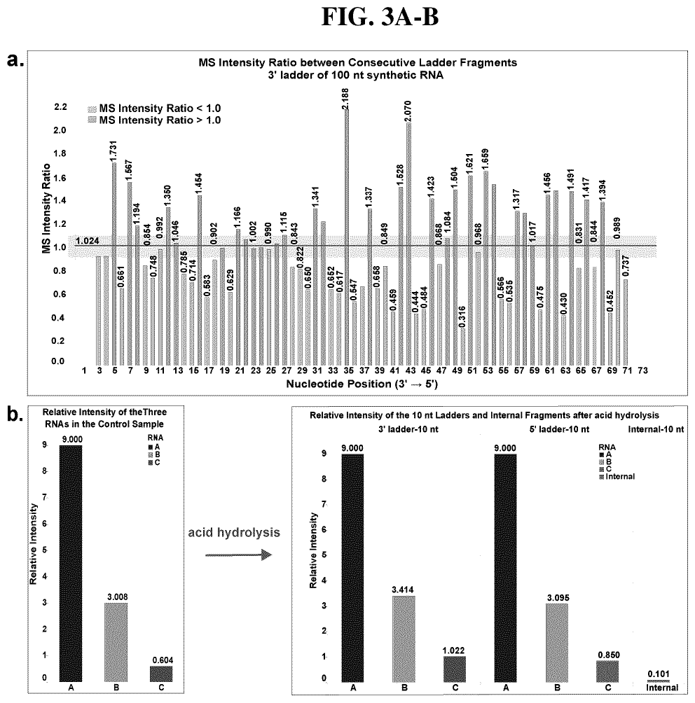

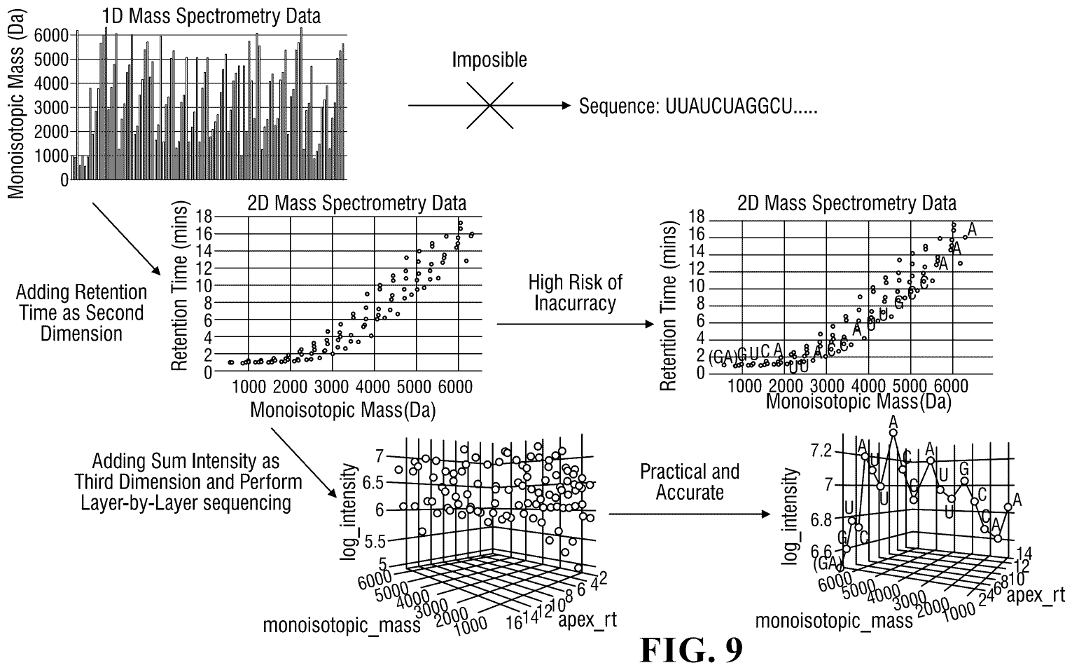

The initial relative intensities of three original RNA molecules and the relative intensities of their respective 10 nt 5′ and 3′ ladder fragments, as well as the relative intensity of the 10 nt internal fragments are depicted in FIG. 3B. After acid hydrolysis, the intensity ratio of ladder fragments from different original RNAs roughly follows the intensity ratio of their original RNAs in the control sample (˜9:3:1), whereas, the internal fragments have much lower intensities compared to all the ladder fragments, including the ladder fragments from the least abundant original RNA. Such fragmentation pattems of RNA molecules in a mixture facilitate the precise and fast data separation of ladder fragments based on the intensity, mass, and retention time (FIG. 9).

As an additional step, following acid hydrolysis of the RNA, a novel Nested Algorithm, is employed to identify all the ladder fragments originated from one parental RNA sequence in the complex LC-MS data of a mixed RNA sample and to further de novo read out each RNA sequence. The provided algorithm effectively leverages the 3D relationship of mass, intensity, and retention time, which are generated simultaneously during LC-MS measurement and are intrinsically related to each RNA or fragment, providing a robust tool to separate data points of ladder fragments into different groups for each RNA and generate its sequence de novo. Specifically, the algorithm segregates data points of consecutive ladder fragments from the same parent RNA based on the original RNA abundance order into a 3D-mass-intensity-retention time layer. Within each laver, a short RNA sequence can be read by base-calling each nucleotide from mass differences between consecutive ladder fragments. The provided method efficiently handles MS data separation and separate different RNA sequences on silica, eliminating the need for complex physical sample separation steps.

In an aspect of the provide sequencing method, the Nested Algorithm initiates the 1^{st} layer by identifying the data point (data point A) with a mass lower than the parent RNA and has the highest signal intensity (i.e., the most intense potential ladder fragment). Furthermore, data point A is likely associated with a ladder fragment from the most abundant RNA in the sample. Starting from point A, a base calling identifies the subsequent data point (data point B) with an added mass equivalent to one nucleotide. This point B likely corresponds to a consecutive ladder fragment from the same RNA, recording as an additional nucleotide in the RNA sequence. Once data point B is identified, the process continues iteratively to determine subsequent data points of RNA ladder fragments.

The searching for the next data point follows four criteria. (i) alignment of mass differences with the searching library, encompassing four canonical nucleotides and RNA modifications; (ii) fitting appropriate retention time (t_{R}) differences into a 2D t_{R} versus mass sigmoidal curve; (iii) selecting the match with highest intensity in cases of multiple matches; and (iv) ensuring intensities within the same order of magnitude for consecutive ladder fragments.

This iterative process continues until no further data points can be found to the mass increase direction. Upon reaching this point, the base calling reverts to data point A, searching for a data point likely belonging to the preceding ladder fragment of data point A from the same RNA, containing one less nucleotide. The searching in the mass decreasing direction similarly follows the above four criteria and persists until no more data points can be found. The algorithm persists in processing the remaining data to produce additional arrays of data points belonging to different parent RNAs. Typically, the order of the generated layers of data points aligns with the intensity order of original RNAs in the sample. The disclosed process allows the identification of all the ladder fragments and produces short RNA sequence readouts de novo in each layer.

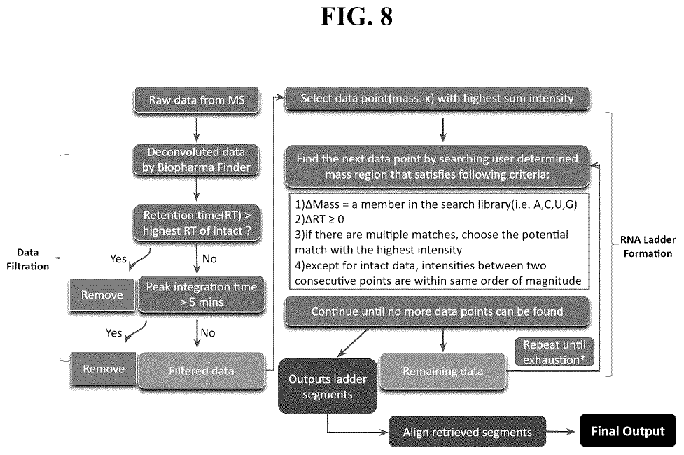

Advantageously, the herein disclosed sequencing method produces sequencing data within a few minutes once the deconvoluted MS data files are available, making it ideal for real-time quality control of therapeutic RNA products in the pharmaceutical industry. FIG. 9 illustrates the 3D data separation strategy of the Nested Algorithm. The flowchart of the Nested Algorithm is shown in FIG. 8.

As an additional step in the provided sequencing method, fragment sequences are assembled to generate the full sequence of RNA. In complex RNA samples, multiple short RNA sequences are read out from each layer as described above. This occurs because the original search library only contains the nucleotides A. G. C. and U and a few of the many known nucleotide modifications. To construct complete RNA sequences, the short sequences are aligned and assembled by considering nucleotide positions and connections inferred from mass differences involving one or more nucleotide combinations. This is possible because the MS sequencing data also carries location information, since the length of an original RNA molecule is approximated by dividing its monoisotopic mass by 320 Da.

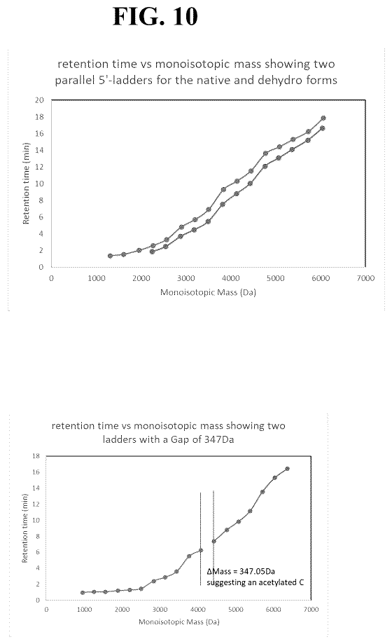

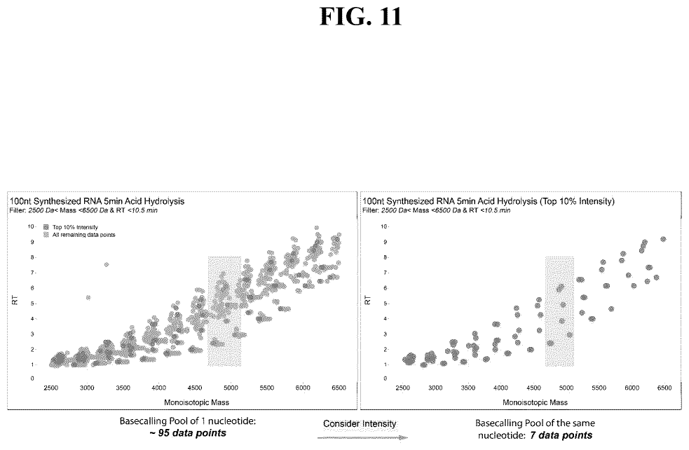

Starting with the most intense sequence readout #1, it is aligned with its parallel sequences (arising from partial modifications or metal adducts) characterized by constant mass differences at every nucleotide position corresponding to the same sequence. In an embodiment, bidirectional readout sequences (reading in either 5′→3′ or 3′→5′ directions) are also taken into consideration. The sequence assembly stage also fills the “gaps” caused by modifications not contained in the searching library, including certain rare modifications like 5-carboxymethylaminomethyl-2-geranylthiouridine (cmnm⁵ges²U)⁸. Examples of parallel sequences and gap between sequences are shown in FIG. 10. The publicly available MassSum algorithm⁹ (see. U.S. patent Ser. No. 17/235,621) segregates the paired ladder fragments for each RNA sequence and groups short sequences. Prior input of target RNA sequences can be helpful but is not required in the sequence assembly. The final results not only verify the target sequence but also reveal unwanted co-existing RNA sequences.

In an embodiment, to verify the credibility (whether the sequences are truly from original RNAs molecule in the mixture) of the automatic read out sequence segments, a scoring system is provided, which examines the de novo sequences using the positioning of ladder fragments and the length of the sequence segment. Specifically, using several criteria one can evaluate the accuracy of the sequence outcome: an agreement with the references, full coverage of an RNA, identification of parallel sequences, depth, and the intensity and length of the ladder sequences. In one aspect, if the obtained sequences agree with the references, the accuracy score is 100 and if mismatches are found the percentage is deducted. If full coverage of an RNA is achieved, the accuracy score is 100; otherwise, a percentage is deducted based on the number of missing positions. Parallel sequences, derived from data points with fixed mass differences (metal adducts, partial modifications), independently sequenced by the nested algorithm, reduces the probability of random false positive read outs. Some common mass differences between parallel sequences and their explanations are shown in Table 8. Longer segment sequences with higher mass values and MS intensity are more likely to be from a parent RNA in the original mixture. In addition, higher MS intensity contributes to a more reliable outcome, while a decrease in the internal fragment region may affect reliability due to increased base calling false positive rates.

In an embodiment, a provided Sequence Segment Scoring System may be used for quantitative evaluation of sequencing accuracy, in cases where a reference sequence is not available, and is used to calculate the probability of false positives at each position based on the base calling pool size and the probability of a random appearance of specific combinations of A, C, G, and U. If a specific A, C, G, and U is not available, it utilizes the maximum false positive from the most common combination to estimate the highest possible false positive value.

For a sequence segment read from the Nesting Algorithm covering positions #h to #k, the corresponding data points to the RNA's ladder fragments at #h−1 to #k are assigned. The false positive for the existence of all k−h+1 data points due to randomness is calculated as the product of false positive values at all k−h+1 positions. The accuracy score of the sequence is then defined as 100 times (1 minus the product of these false positives). A score of 95 means there is 5% chance this segment sequence is the result of the random distribution of the data.

As an illustrative example, if the base calling pool size is 90, a sequence covering positions #5-14 has a score of 54, and a sequence covering positions #15-24 has a score of 99. If the base calling pool size is 200, then the corresponding scores are 6 and 69, respectively. Larger base calling pool sizes increase data complexity and decrease the score. Higher positions in the sequences and longer sequences result in higher scores.

In an embodiment, the presently provided sequencing method may be used to detect impurities in synthetic RNA sequences identified using the Nested Algorithm. For example, in the RNA samples before acid hydrolysis, one can detected RNA impurities (intact level) likely introduced as by-products during chemical synthesis. In such a case, ladder fragments and sequences associated with some potential impurities (ladder level) may be extracted, considering the impurities observed at the intact level before acid hydrolysis. To assess the credibility of these sequences (some of which are unrelated to the target RNAs. i.e., undesired impurities), scores are assigned using the scoring system described above. Sequences obtained from the layers during the layer-by-laver sequencing by the nested algorithm may be proven as unrelated sequences and unrelated ladder fragments to an RNA after comparing with the theoretical sequences and theoretical mass of ladder fragments. Besides using the smoothness of the t_{R}-monoisotopic sigmoid curve and intensity similarities between consecutive ladder fragments to help validate the ladder fragment belongings, a score of larger than 90 (based on probability and ladder positioning) can also help to assess whether ladder fragments should be grouped together.

Accordingly, the nested algorithm coupled with the scoring system disclosed herein provides a fast and efficient RNA sequencing framework to de novo sequence RNAs from mixtures, assess and assemble sequence segments, as well as quantitatively examine the impurities in the sample.

The present disclosure provides a kit for use in generating the sequence of one or more RNA molecules, detecting the presence, identity, location, and quantity of RNA nucleotide modifications, and/or possible impurities, on said one or more RNA molecules, said kit comprising one or more components for performance of a method comprising one or more of the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) processing of data through use of the disclosed nested algorithm coupled with the scoring system thereby generating the sequence of one or more RNA molecules, detecting the presence, identity, location, and quantity of RNA nucleotide modifications and/or the presence of possible impurities.

The present disclosure provides a computer-implemented method for determining an order of nucleotides and/or modifications of an intact RNA or a controlled enzymatically cleaved RNA molecule, wherein the method includes: receiving/exporting liquid chromatography-mass-spectrometry (LC-MS) data of an RNA sample, the LC-MS data including but not limited to a mass (e.g., m/z, monoisotopic mass, average mass), charge states, retention time (t_{R}), Hight, width, volume, relative abundance, and quality score (QS); filtering the LC-MS data based on mass, the filtering including removing masses smaller than a predetermined size; analyzing the filtered LC-MS data, to determine a plurality of RNA sequences, and processing the LC-MS data through use of the disclosed nested algorithm and the scoring system provided herein.

In an embodiment, provided is a non-transitory computer-readable medium storing instructions that, when executed by a processor, cause the processor to perform a method for generating the sequence of one or more RNA molecules and detecting the presence, identity, location, and quantity of RNA nucleotide modifications on said one or more RNA molecules, the method comprising the steps of (i) identifying a specific chemical moiety associated with the RNA thereby imparting an identifiable property on the RNA (ii) controlled fragmentation of the RNA to form 5′ and 3′ MS ladder fragments. (iii) mass measurement of resultant degraded RNA samples containing RNAs and their degraded fragments; and (iv) processing the LC-MS data using the disclosed nested algorithm and the scoring system provided herein.

Any of the herein described methods, programs, algorithms or codes may be converted to, or expressed in, a programming language or computer program. The terms “programming language” and “computer program.” as used herein, each include any language used to specify instructions to a computer, and include (but is not limited to) the following languages and their derivatives: Python, Assembler, Basic, Batch files, BCPL, C, C+, C++, Delphi, Fortran, Java, JavaScript, machine code, operating system command languages, Pascal, Perl, PL1, scripting languages. Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

### Example 1

### Materials and Methods

Reagents. LC-MS grade methanol, triethylamine (TEA), 1,1,1,3,3,3-hexafluoro-2-propanol (HFIP) and analytical grade formic acid were purchased from Thermo Fisher Scientific (Waltham, MA, USA). HPLC grade water was purchased from Sigma-Aldrich (Burlington, MA. USA). Synthetic RNAs (about 10 nmol) were purchased from Integrated DNA Technologies (IDT) (Coralville, Iowa. USA).

Hydrolysis reaction. Synthetic RNAs (1-10 pmol) were vortexed with RNase free water and transferred to 0.5 mL eppendorf tubes. Identical volumes of formic acid were mixed with RNA solutions at room temperature and then transferred to a thermal cycler. The RNA mixture was kept at 40° C. for 0.5-10 minutes, respectively before quenched in dry ice. The sample was then dried down using an Eppendorf Vacufuge plus instrument (Eppendorf, Enfield, CT, US).

Reversed phase liquid chromatography A DNAPac RP column (4 um, 2.1×100 mm) was purchased from Thermo Fisher (Waltham, MA. USA) and installed in a Vanqiush Horizon UHPLC system (Thermo Fisher. Waltham, MA, USA). Mobile phase A was prepared with 15 mM TEA and 50 mM HFIP buffers in aqueous solution. Mobile phase B was prepared at the ratio of 50:50 water/methanol with buffer 7.5 mM TEA and 25 mM HFIP. During LC analysis, the column temperature was maintained at 60° C. and the flow rate was set at 0.3 mL/min. The composition of mobile-phase B was started at 5% and increased to 10% in 5 min. Then it was increased to 25% in 20 min and again raised to 50% in another 15 min. Five microliters of RNA solutions were injected by the autosampler without dilution.

High-resolution mass spectrometry. A heated electrospray ionization (HESI) probe was installed in an orbitrap Exploris 240 mass spectrometer (Thermo Fisher, Waltham, MA, USA) coupled to LC. The capillary voltage was set at 3.4 kV in negative mode. Sheath and aux gas were set to 45 Arb and 10 Arb. The temperature of the ion transfer tube and vaporizer were 320° C. and 275° C., respectively. Other parameters were set as follows: 500 ms scan time, 300% normalized AGC target, 3 microscans, 70% RF lens and 300 ms maximum injection time. Full scans were performed at m/z 600-2000 range and at 240,000 resolution.

Software and data processing. The coupled LC and orbitrap instruments were controlled by Chromeleon 7.1 and Xcalibur 4.2, respectively. TIC and MS raw data obtained from LC-MS analysis was processed by BioPharma Finder 5.0/5.2 and deconvoluted by the embedded Xtract algorithm.

The development of the Nested Algorithm disclosed herein is rooted in the principles governing chemical reactions occurring during the acid hydrolysis of RNA molecules in the mixture. The core concept of the LC-MS based RNA sequencing is to assign LC-MS signals to fragments generated through controlled formic acid hydrolysis. The identity and location of any nucleotide from the original RNA are determined by the mass difference of consecutive ladder fragments.

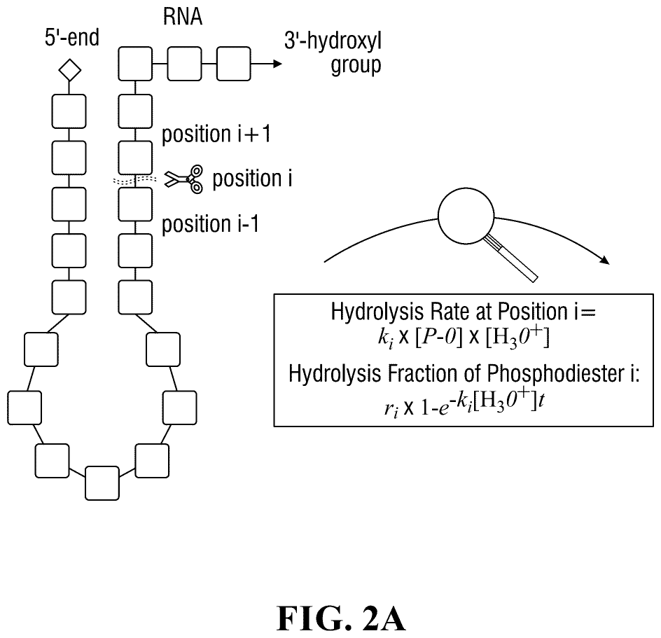

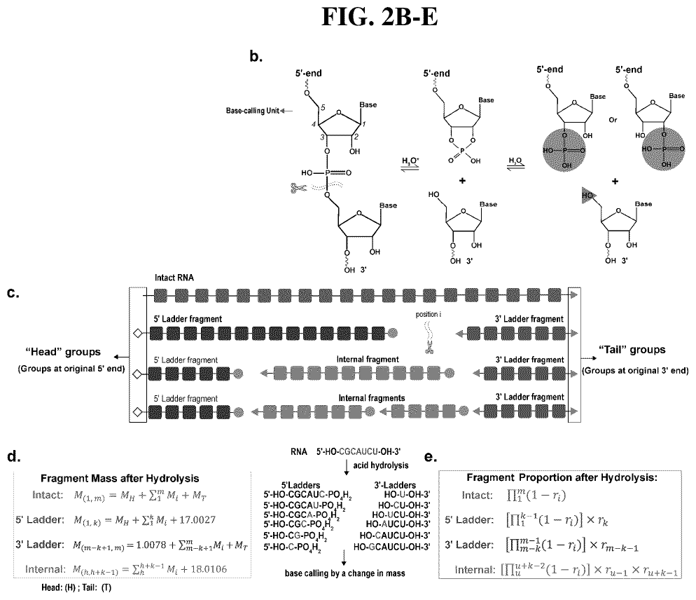

To establish the foundation of mass spectrometry-based sequencing and ensure consistency in method development, it is crucial to unambiguously define terms. A nucleotide within an RNA comprises one unit of phosphate-ribose diester through the hydroxyl groups on the ribose's 3 and 5 positions, with a nucleobase attached to the ribose from the 1 position (see FIG. 2B). It is further defined that the phosphate within this nucleotide is attached to the 3′-carbon. The masses of the parent RNA and its fragments are expressed as a function: M (a, b), where a and b represent the original positions of the first and last nucleotides in the fragments from the parent RNA or the parent RNA itself.

After a single hydrolysis, the P—O ester bond at the ribose's 5-position is cleaved, resulting in two fragments. The one with the original head now possesses a new hydroxyl group (—OH) tail, while the one with the original tail has a new hydrogen (—H) tail. Post-hydrolysis of the original RNA, three types of fragments can be produced (FIG. 2C):
5′-Ladder Fragments: These are fragments with the original 5′ end (head).
3′-Ladder Fragments: Fragments with the original 3′ end (tail).
Internal Fragments: Fragments without the original 5′ and 3′ ends, resulting from multiple cleavages on the original RNA molecule.
   The masses of the parent RNA and its fragments can be expressed as (FIG. 2D):

$$Parent RNA with length m : M ( 1 , m ) = {M}_{Head} + {M}_{Tail} + {\sum }_{1}^{m} {M}_{i}$$

$${5}^{′} - ladder fragment with length k ∈ ( 1 , m - 1 ) : M ( 1 , k ) = {M}_{Head} + 17.0027 + {\sum }_{1}^{k} {M}_{i}$$

$${3}^{′} - ladder fragment with length k ∈ ( 1 , m - 1 ) : M ( n - k + 1 , m ) = {M}_{Tail} + 1.0078 + {\sum }_{m - k + 1}^{m} {M}_{i}$$

$$Internal fragment with length k ∈ ( 1 , m - 2 ) : M ( h , h + k - 1 ) = 18.0206 + {\sum }_{h}^{h + k - 1} {M}_{i}$$

Mᵢ is the monoisotopic mass of the nucleotide (canonical or modified) at position i from the parent RNA, M_{Head}, M_{Tail}, 17.0027, 1.0078, and 18.0106 are the monoisotopic masses of the head, tail, —OH, —H and H₂O involved. The therapeutic synthetic RNAs generally have-H as the head (at 5′ end) and —OH (instead of a PO₄H₂) at the 3′ end. The M_{Head} for therapeutic synthetic RNAs is 1.0078 and M_{Tail} is −79.9685. The unit of all masses is Dalton or amu. Throughout the specification a dotted box represents a space.
Once consecutive ladder fragments are assigned, the nucleotide mass at the corresponding position is determined by taking the mass differences of these two consecutive ladder fragments. For example, the mass of the k^{th} nucleotide from the parent RNA can be calculated by:

$$Mk = M ( 1 , k ) - M ( 1 , k - 1 )$$

Thus, successful sequencing of RNA relies on the successful assignments of the ladder fragments originating from different original RNAs. To make accurate and unambiguous assignments of the LC-MS data to the corresponding fragments generated from acid-hydrolysis, the hydrolysis kinetics were examined.

### Fragmentation of RNA Through Controlled Acid Hydrolysis

The controlled acid hydrolysis generates RNA fragments by cleaving the phosphodiester bonds at varying nucleotide positions. Given an original RNA with m nucleotides, there are 2(m−1) ladder fragments and a total of

$$\frac{( m - 2 ) ( m - 1 )}{2}$$

internal fragments that can be generated theoretically. Using the site-specific P—O hydrolyzed fraction rᵢ values from Equation (4), the amount of the unhydrolyzed intact RNA and each type of fragment after acid hydrolysis can be expressed. For an RNA molecule with m nucleotides and an initial amount C₀, the unhydrolyzed intact RNA can be expressed as C₀ Π₁^{m-1} (1-rᵢ). The product. Π₁^{m-1} (1-rᵢ), is the probability that none of the m−1 site is hydrolyzed. Similarly, the amounts of ladder fragments with k nucleotides (k∈(1, m−1)), from nucleotide #1 to #k, or #m−k+1 to #m, respectively, C₀ (Π₁^{m-1} (1-rᵢ))rₖ or C₀ (Π₁^{m-1} (1-rᵢ))rₘ₋ₖ, where (Π₁^{m-1} (1-rᵢ))rₖ is the probability that none of the k−1 sites from #1 to #k−1 is hydrolyzed, but the #k site is hydrolyzed. Moreover, for internal strands with k nucleotides (k ∈(1, m−2)) from #h to #h+k−1, the amount can be approximately expressed as C₀(Π₁^{m-1} (1-rᵢ)rₕ₋₁rₕ₊ₖ₋₁, assuming the rate constants of hydrolysis at each sites do not change during the reaction. The formula for calculating the proportions of the unhydrolyzed intact RNA molecules and different types of fragments using the site-specific fraction of hydrolysis are shown in FIG. 2E.

To collect optimal data, three separate LC-MS runs are conducted on each RNA sample. To obtain information on the total number of different RNA molecules, their modifications, and their relative abundances that allow to set the threshold for, we performed a control run 1 by directly measuring 5% of the sample intact without acid hydrolysis. To gather insights into optimal RNA hydrolysis conditions for efficiently producing well-define ladders of the target RNA samples, control run 2 was performed using the same sample quantity but subjected to acid-hydrolysis for 5 minutes in 40° C. These conditions were based on previous empirical work but not yet optimized for each RNA sample 7. LC-MS configurations and running parameters were also optimized accordingly to effectively measure well-define ladders for MS sequencing of the target RNA samples.

Total Number of Fragments from a Parent RNA with Length m
There is one unhydrolyzed intact parent RNA, 2 (m−1) ladder fragments with length 1 to m−1, and

$$\frac{( m - 1 ) ( m - 2 )}{2}$$

internal fragments. For any given number k, (1<k<m), there are 4 ladder fragments and m−k−1 internal fragment(s).

### Total Number of Sequences (Canonical Nucleotides Only)

For an RNA with length m, the total number of all possible sequences is 4^{m}.

### Total Combinations of Sequences (Canonical Nucleotides Only)

For an RNA with length m, the total number of all possible combinations of ACGU arrangements is

$$( \frac{m + 3}{m} ) = ( \frac{m + 3}{3} ) = \frac{( m + 1 ) ( m + 2 ) ( m + 3 )}{6}$$

Number of Sequences from a Typical Combination AaCcGgUu

a, c, g, u are non-negative integers as the number of the corresponding nucleotide, and a+c+g+u=k, the number of total possible sequences is

$$\frac{k !}{a ! c ! g ! u !}$$

Random Chance of the Occurrence of AaCcGgUu Among all RNAs with Length k (k=a+c+g+u, Canonical Only)

Statistical chance of its occurrence is

$$\frac{k !}{a ! c ! g ! u ! {4}^{k}}$$

### Fraction of Hydrolysis Dependency on Hydrolysis Time.

rᵢ=1−e^{-k}ᵢ′t≅kᵢ′t, when kᵢ′t is small (<0.05), in our experiments, rᵢ is always controlled less than 0.05. With this approximation, rᵢ is directly proportional to hydrolysis time t.

### Random Error of Fragments Probability

The probability of formation for ladder fragment is: (1-rₐᵥ)^{k-1}rₐᵥ, and eᵣ=0.2 is relative random error (relative standard deviation) of rₐᵥ. The relative error of (1-rₐᵥ)^{k-1}rₐᵥ, is

$$\frac{d [ {( 1 - {r}_{av} )}^{k - 1} {r}_{av} ]}{{dr}_{av}} ( {r}_{av} \cdot {e}_{r} ) / [ {( 1 - {r}_{av} )}^{k - 1} {r}_{av} ] = \frac{\vert 1 - {kr}_{av} \vert}{1 - {r}_{av}} {e}_{r}$$

Optimal rₐᵥ for ladder fragment with length k
Optimal r_{Av} for Ladder Fragment with Length k

The best rₐᵥ for ladder strands with length k after hydrolysis can be estimated by taking the first derivative of its fraction expression vs rₒᵥ, the condition of optimal fraction happens when

$$d ( {( 1 - {r}_{av} )}^{k - 1} {r}_{av} ) / {dr}_{av} = {( 1 - {r}_{av} )}^{k - 1} - ( k - 1 ) {( 1 - {r}_{av} )}^{k - 2} {r}_{av} = 0$$

thus, when

$${r}_{av} = \frac{1}{k} ,$$

the fractions of ladder fragments with length k reach maximum value. This maximum fraction can be expressed as

$${( 1 - {r}_{av} )}^{k - 1} {r}_{av} = \frac{{( k - 1 )}^{k - 1}}{{k}^{k}}$$

### Optimal Hydrolysis Time-Kinetics, Probability, and Statistical Studies of RNA Acid-Hydrolysis Reactions.

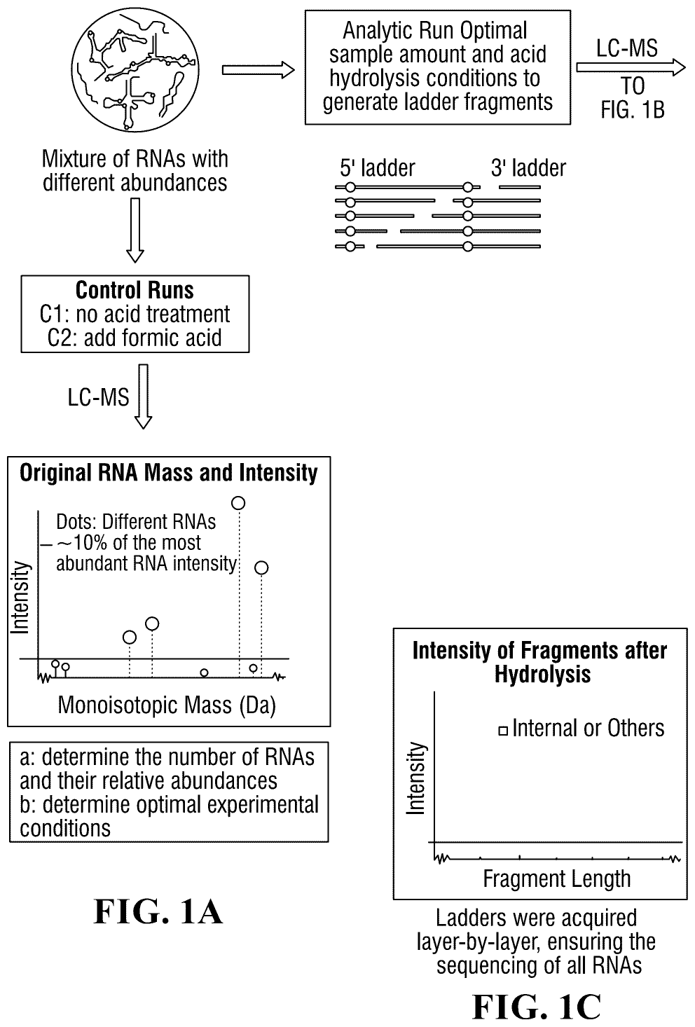

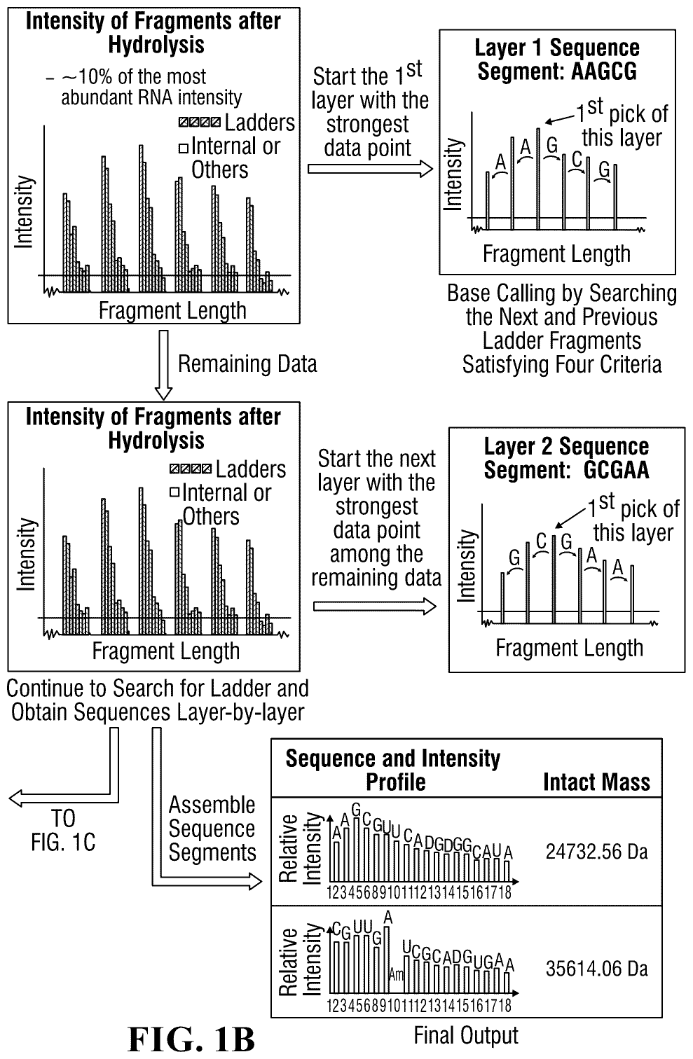

A shorter hydrolysis time results in a smaller rₐᵥ value, yielding lower fractions of all fragments (calculated from the formula shown in Table 1), regardless of ladder or internal fragments, along with a lower MS intensity. Nevertheless, short hydrolysis time indeed results in a desirable ladder-to-internal fragment intensity ratio for separating ladder fragments from internal fragments. This is important for sequencing RNAs with very weak relative intensities, where very small rₐᵥ values can cause their ladder fragments to have higher intensity than the internal fragments from much more abundant RNAs. On the other hand, a long hydrolysis time leads to a large rₐᵥ value and a low fraction of long fragments (with more nucleotides) after acid hydrolysis. Previous approaches involved dividing the sample into distinct portions, hydrolyzing them for varying durations, and subsequently pooling them together for LC-MS measurement to balance fragment distribution. However, by utilizing an optimal acid hydrolysis time, fractions of ladder fragments are increased by 10-50% compared to the splitting-pooling approach. Detailed calculations and comparisons of fragment fractions from hydrolysis at different rₐᵥ values are available in the Supporting Information (Table 1). The new workflow (FIG. 1) offers advantages such as streamlined sample handling, providing more intense MS data, and improved ladder/internal fragment intensity ratios.

90% of the RNA sample is subjected to acid treatment under optimal conditions. This analytical run provides essential data for the nested algorithm to sequence different RNAs in mixtures. The generated raw LC-MS data is then processed by BioPharma Finder (v5.0-5.2, Thermo Scientific) to report monoisotopic mass, retention time, intensity among other information for all components as a formatted multi-dimensional dataset. The Nested Algorithm is then applied to read out RNA sequences layer by layer for sequence alignment and assembly.

Approximate Probabilities of Formation of Each Type of Fragment from the Respective Parent RNA Hydrolysis Using the Average Fraction of Hydrolysis rₐᵥ

It is unrealistic to use the site-specific fraction of hydrolysis values ri, especially when the sequence of the RNA is not determined. As an approximation, one can use the average fraction of hydrolysis per site, rₐᵥ, and its relative uncertainty, er, to estimate the amount of different types of fragments and the amount of intact RNAs after acid hydrolysis. Using rₐᵥ to replace rᵢ values in the formula from the last section, the ladder fragments and internal fragments amounts can be approximated in the table below. The relative uncertainties were calculated based on statistical rules.

Approximated amounts and their relative uncertainties for ladder and internal fragments using rₐᵥ and er.

| Type of fragment | Amount derived from | | Amount approximated | Relative uncertainty |
|---|---|---|---|---|
| with length k | site-specific fraction rᵢ | | from rₐᵥ | from rₐᵥ and eᵣ |
| 5′-ladder fragment | C₀(Π₁^{k-1} | (1 − rᵢ))rₖ | C₀(1 − rₐᵥ)^{k-1}rₐᵥ | $\frac{\vert 1 - {kr}_{av} \vert}{1 - {r}_{av}} {e}_{r}$ |
| 3′-ladder fragment | C₀(Πₘ₋ₖ₊₁^{m-1} | (1 − rᵢ))rₘ₋ₖ | C₀(1 − rₐᵥ)^{k-1}rₐᵥ | $\frac{\vert 1 - {kr}_{av} \vert}{1 - {r}_{av}} {e}_{r}$ |
| Internal fragment | C₀(Πₕ^{h+k-2} | (1 − rᵢ))rₕ₋₁rₕ₊ₖ₋₁ | C₀(1 − rₐᵥ)^{k-1}rₐᵥ² | $\frac{\vert 2 - ( k - 1 ) {r}_{av} \vert}{1 - {r}_{av}} {e}_{r}$ |

Ladder Fragments Exhibiting MS Intensities Much Higher than Internal Fragments.

Taking the formula in Table 1, the ratio of the probability of formation between a ladder fragment and an internal fragment with same length k, can be calculated as:

$$\frac{ladder fragment intensity}{internal fragment intensity} = \frac{{( 1 - {r}_{av} )}^{k - 1} {r}_{av}}{{( 1 - {r}_{av} )}^{k - 1} {r}_{av}^{2}} = \frac{1}{{r}_{av}} .$$

Since rₐᵥ is very small, typically less than 0.05, the intensity ratio between ladder and internal fragment is at least greater than 20. This ratio can be adjusted even higher by controlling the rₐᵥ to a smaller value, which thus contributes to the accurate separation of ladder fragments from internal fragments based on intensity.
Determination of the rₐᵥ relative uncertainty (er) of rₐᵥ
The relative uncertainty of the average fraction of hydrolysis is hard to evaluate. Therefore, we approximate it from the ratio of the MS intensities of consecutive ladder fragments.

$$\frac{MS intensity of ladder fragment with length k}{MS intensity of ladder fragment with leng k - 1} = \frac{{q}_{k} {C}_{0} ( {\prod }_{1}^{k - 1} ( 1 - {r}_{i} ) ) {r}_{k}}{{q}_{k - 1} {C}_{0} ( {\prod }_{1}^{k - 2} ( 1 - {r}_{i} ) ) {r}_{k - 1}} = \frac{{q}_{k} ( 1 - {r}_{k - 1} ) {r}_{k}}{{q}_{k - 1} {r}_{k - 1}} ≅ \frac{{r}_{k}}{{r}_{k - 1}}$$

Where qₖ and qₖ₋₁ are MS response factors, which are approximately equal for fragments with similar length, and rᵢ values are relatively small, <0.05. Thus, the consecutive ladder fragments intensity ratio should be approximately equal to the consecutive site-specific fraction of hydrolysis ratio FIG. 3A shows the ratios of consecutive 3′-ladder fragments from a synthetic 100-nucleotide RNA spanning from #3 to #71. These ratios varied between 0.3 and 2.2, with an average of approximately 1, indicating that even though there are variations in these rᵢ values across different sites, their distribution does not significantly span a wide range. Based on this approximation, we have calculated the relative uncertainty eᵣ for hundreds of RNA molecules with lengths of between 20 and 100 nucleotides, and the values of eᵣ are between 10 and 30%. The mathematical foundations lead to the two key characteristics described in the method workflow section for nested algorithm development.
Ladder Fragments Exhibiting MS Intensities Nuich Higher than Internal Fragments.
Taking the formula in Table 1, the ratio of the probability of formation between a ladder fragment and an internal fragment with same length k, can be calculated as:

$$\frac{ladder fragment intensity}{internal fragment intensity} = \frac{{( 1 - {r}_{av} )}^{k - 1} {r}_{av}}{{( 1 - {r}_{av} )}^{k - 1} {r}_{av}^{2}} = \frac{1}{{r}_{av}} .$$

Since rₐᵥ is very small, typically less than 0.05, the intensity ratio between ladder and internal fragment is at least greater than 20. This ratio can be adjusted even higher by controlling the ray to a smaller value, which thus contributes to the accurate separation of ladder fragments from internal fragments based on intensity.

### Choice of Searching Library

The nested algorithm is designed to use a flexible searching library, and the approach of employing a three-level searching library is a strategic choice based on the complexity of the sample RNA to balance sequencing accuracy and efficiency. The searching library consists of monoisotopic mass values for canonical and modified nucleotides, and in the base calling step, the mass difference between each candidate and the current data point must match those in the searching library with a certain tolerance (e.g., 0.05 Da, adjustable).

The three levels of searching library are defined as follows:
Level 1: Canonical nucleotides only (A. C. G and U).
Level 2: A. C. G. U and a few common modifications, such as dihydrouridine and methylated A/C/G/U.
Level 3: A. C. G. U and all reported modifications.
   Depending on the characteristics of the target sample, different libraries may be used for sequencing during the base calling stage. It's important to note that the differences in outcomes from the three-level searching libraries are reflected in the length of the ladder sequences obtained. Ladder sequences obtained from levels 1 and 2 are typically shorter, requiring an additional assembly step for further analysis and the addition of potential RNA modifications not included in these libraries.
   Specifically, when using levels 1 and 2 libraries, possible “gaps” caused by the absence of certain modifications during the nested base calling process may be observed between ladder sequence segments. On the other hand, directly using the Level 3 search library may generate some “unusual” sequences with high frequencies of rare modifications, making it more suitable for very pure samples, in which the intensity criteria alone can eliminate all possible mismatches (detailed later in false positive from intensity section).
   This strategy allows for flexibility in adapting the searching library to specific characteristics of the target RNA sample, ensuring both accuracy and efficiency in the sequencing process.

### Retention Time Consideration

The incorporation of retention time as a criterion in addition to mass difference and intensity is a valuable step in ensuring accurate sequencing. Generally, longer fragments exhibit increased retention times, unless specific modifications significantly alter the polarity of the fragments generated at the hydrolysis site. By considering retention time alongside mass difference and intensity, the sequencing process gains an additional layer of accuracy. This multi-criteria approach helps mitigate false positives in ladder fragments and reduces the likelihood of reading incorrect sequences.

### Base Calling Accuracy Analysis

In the nested algorithm, three aspects of the target data points are considered in the order of mass, intensity, and retention time. The potential for wrong assignment of data points arises from a combination of three random events: (1) the mass value of a random strand satisfying the search library; and (2) the MS intensity of this data point being higher than the correct assignment; and (3) retention time trend being correct. Considering the size and complexity of the hydrolyzed data set, probability and statistical tools are employed to estimate the chance of wrong assignment in each base calling step. This approach allows for a quantitative assessment of the reliability of the algorithm in handling any datasets, providing insights into the potential errors that may occur during the sequencing process.

The accuracy of base calling is influenced by the size of the base calling pool, defined as a set of data points with mass values 300-500 Da different from the current data point. Data points in the base calling pool come from fragments with the targeted length and they are hydrolysis products from all parent RNAs in the sample. Given that internal fragment intensity is much lower than ladder fragment intensity, applying a relative intensity filter (e.g., at 3*rₐᵥ level) effectively removes most internal fragments.

If there are n RNA species in the sample, the size of the base calling pool is approximately 2n. Additionally, retention times and exact monoisotopic mass differences are utilized to distinguish between 3′ and 5′-ladder fragments. Despite having similar mass and intensity, 5′-ladder fragments typically exhibit higher retention times due to the extra phosphate groups they carry. In addition, if the masses of a 5′-ladder fragment and a 3′-ladder fragment are numerically equal on the unit level, the 5′-ladder normally is 0.1 Da higher than the 3″-ladder fragments. This is due to the mass difference between a phosphorus atom (30.9738 Da) and its replacement, such as C₂H₇ (31.0548 Da). The 0.05 Da tolerance level used in base calling can prevent the mismatch of 5′ and 3′-ladder fragments. Considering retention times and exact masses further reduces the size of the base calling pool to n. In the next section, one estimates the probability of misassignment under each circumstance.

False Positive from Mass

Estimating the probability of a random strand satisfying the masses targeted in the base calling step (false positive) is indeed a crucial aspect of ensuring the accuracy of sequencing process. Given the dependence on the exact combination of nucleotides and the position of the base calling, calculating this probability for each mass value can be computationally overwhelming. Two examples of detailed base calling mismatch probability at 5158.676 Da and 2565.316 Da, using level 1 and level 2 searching library are shown in Table 9.

To manage this complexity, it is beneficial to calculate the maximum probability value. The maximum value is defined as 3 times the probability of a random strand with length k having the most common combination of ACGU. An upper limit for false positives allows for a more manageable and efficient assessment of the potential for incorrect assignments.

As an example, in the worst-case scenario, specifically at base calling position #9 (searching for a data point corresponding to a ladder fragment with 9 nucleotides long) using the canonical ACGU masses as the search library (level 1 library), the maximum probability to give a false positive is 8.7%. This value is 3.4% at position #18. For Level 2 and Level 3 libraries, the upper limits for false positive estimates are higher, calculated as 2 and 3 times, respectively, of the Level 1 library.

An Excel file (SI-max false positive from mass calculator) is available for calculating all these maximum false positive values. This tool allows for a comprehensive and systematic assessment of the potential false positives across different base calling positions and search libraries.

False Positive from Intensity

Mass match is only one of the three criteria used in base calling, and intensity is another important factor. During base calling, if multiple mass matches happen, priority is given to the one with higher intensity. We need also to estimate the probability of a fragment from a less abundant strand having a higher MS intensity than the one from a more abundant strand.

Assuming two parent RNA, whose amounts are C₁ and C₂, (C₁>C₂) respectively. The MS intensities of the 5′-ladder fragments with length k after hydrolysis are q₁ₖC₁(1-rₐᵥ₁)^{k-1}rₐᵥ₁, and q₂ₖC₂(1-rₐᵥ₂)^{k-1}rₐᵥ₂, with relative error of

$$\frac{\vert 1 - {kr}_{av} \vert}{1 - {r}_{av}} {e}_{r} ,$$

while q₁ₖ and q₂ₖ are the signal response factor for the corresponding length k 5′-ladder fragment, q₁ₖ@q₂ₖ. Using the approximation from section 2.2, so that rₐᵥ₁=rₐᵥ₂ and their random errors are independent, the MS intensity difference of these two fragments can be written as q₁ₖ(1-rₐᵥ)^{k-1}rₐᵥ(C₁−C₂), and

$$error = {{q}_{1 k} ( 1 - {r}_{av} )}^{k - 1} {r}_{av} \sqrt{( {C}_{1}^{2} + {C}_{2}^{2} )} \frac{\vert 1 - {kr}_{av} \vert}{1 - {r}_{av}} {e}_{r} .$$

The probability of the MS intensity difference of these fragments less than zero can be calculated using the difference, random error, and statistical tools. Zero is

$$p = \frac{difference}{error} = \frac{{{q}_{1 k} ( 1 - {r}_{av} )}^{k - 1} {r}_{av} ( {C}_{1} - {C}_{2} )}{{{q}_{1 k} ( 1 - {r}_{av} )}^{k - 1} {r}_{av} \sqrt{( {C}_{1}^{2} + {C}_{2}^{2} )} \frac{\vert 1 - {kr}_{av} \vert}{1 - {r}_{av}} {e}_{r}} = \frac{( {C}_{1} - {C}_{2} )}{\sqrt{( {C}_{1}^{2} + {C}_{2}^{2} )} \frac{\vert 1 - {kr}_{av} \vert}{1 - {r}_{av}} {e}_{r}}$$

times away from the mean, the probability can be quantified. For example, is p=2, the probability of the intensity difference less than zero is 0.5-0.477=2.3%.

Mathematically, the amount ratio of C₁ and C₂ can be used in the formula to find p.

$$Let s = \frac{{C}_{1}}{{C}_{2}} , then p = \frac{( s - 1 )}{\sqrt{( {s}^{2} + 1 )} \frac{\vert 1 - {kr}_{av} \vert}{1 - {r}_{av}} {e}_{r}} .$$

As an example, if C₁:C₂=4:3, k=15, rₐᵥ=0.02, and error for fraction of hydrolysis eᵣ=0.2, then p=1.4, then probability of fragment from lesser strand higher than the one from more abundant strand is 8.1%. The values of the amount ratios of different parent RNAs can be read from the MS intensity from Control Run 1. An excel file (SI-max false positive from mass calculator) to calculate any false positive values based on concentration ratios of parent RNAs, rₐᵥ, eᵣ, and base calling position is available in SI. Mismatch probability of some common intensity ratios at selected length using 0.02 as rₐᵥ and 0.2 as its relative error is shown in Table 10.
Expected combined base calling accuracy ratio in one base calling step.
For any base calling step to be wrong, the false data point must satisfy all three judgements. The overall false positive probability for that data point is the multiplication of all three. Since there are multiple data points in the base calling pool, if any data point produces false positive, the base calling step fails to give correct outcome.

Let Fm, Fi, and Fr represent the false positive probability from mass difference, intensity, and retention time, respectively, the overall false positive probability for the corresponding data point is Fm*Fi*Fr. The overall combined base calling accuracy can be expressed as Π₁^{k} (1-Fₘ×Fᵢ×Fᵣ). The values of Fₘ can be estimated from SI-max false positive from mass calculator and they are generally less than 0.1 when the mass of the data point is greater than 2500 Da (8 nt). The values of Fᵢ can be obtained from MS intensity ratios from control run 1 and SI-max false positive from intensity calculator or read from Table 10

An example: There are 50 data points in the base calling pool at mass ˜5000 Da (15 nt) on the 5′-ladder. From control run 1, only 3 parent RNAs have an intensity greater than 50% of the most abundant one, and the ratios are 0.75, 0.67 and 0.50. Fₘ at ˜5000 is 0.044 using Level 1 search library, and Fᵢ values are 0.081, 0.026 and 0.0009, for the three ratios, respectively. Fᵣ is used to eliminate the false positive chance from the opposite 3′-ladder data points, so that Fᵢ=1 for 5′-ladder data points and 0 for 3′-ladder data points. The combined accuracy is (1−0.044*0.081)*(1−0.044*0.026)*(1−0.044*0.0009)=99.53%. The rest 40+ data points have no effect for the combined accuracy since their false positive from intensity Fᵢ is practically 0. This example should show that combined accuracy for base calling is significantly affected by the intensity ratios from parent RNAs in the sample.

Some general guidelines obtained from the accuracy analysis would be beneficial in sequencing different types of RNA samples. Relatively Pure Sample (fraction of the most abundant RNA>70%). False Positive Rate: ˜0.01% based on intensity alone. Best Strategy: Use the level 3 library (ACGU and all reported modifications). Result: Sequence can be read directly without the need for sequence assembly. Mixtures with Close Intensity Ratios (5:4). False Positive Rate: Up to 1.5% at position 9, 0.39% at position 18, using the level 1 library (ACGU). Recommended Strategy: Choose the level 1 library. Result: Full sequences can be obtained after the sequence assembly step. These guidelines provide a starting point for selecting the appropriate library level and strategy based on the intensities profile of the RNA sample. Tailoring the approach to the specific features of the sample helps optimize the sequencing process and ensures accurate results. Factors influencing the accuracy of de novo sequences obtained.

### Definitions

Error rate: (No. of incorrect base calls)/(sequence length)*100%
Sequencing accuracy: (No. of correct base calls)/(sequence length)*100%
Sequencing coverage: (No. of base called nucleotides)/(sequence length)*100%
Sequencing depth: The number of times each nucleotide was read from the RNA ladders, including 3′ and 5′ ladders in native, hydrated, dehydrated, and metal adduct forms.
Sample complexity: The number of RNAs in the sample and their relative intensities.

### Results

Overview of Nested Algorithm-based NGMS-Seq to sequence RNAs from mixtures. The overall workflow of nested algorithm-based NGMS-Seq, illustrated in FIG. 1, includes three essential steps: 1) RNA sample preparation, 2) LC-MS measurement of RNAs intact before and after acid hydrolysis to collect data for MS sequencing and analysis, and 3) data analysis for de novo sequencing of RNA and their modifications using a layer-by-layer nested sequencing algorithm, followed by sequence alignment and assembly.

The intricately designed workflow and nested algorithm draw inspiration from both empirical observations and theoretical considerations of RNA hydrolysis patterns. Key characteristics shaping this design include: 1) the occurrence of diverse types of fragments quantified through the average ratio of hydrolysis per nucleotide, defined as rₐᵥ, which is critical for sequencing and inherently reliant on hydrolysis time, 2) the MS intensity ratio between internal fragments and desired ladder fragments approximates the rₐᵥ value, which can be controlled via well-designed experimental conditions (FIG. 15); and 3) the MS intensity order of desired ladder fragments from different parent RNAs typically aligns with the abundance order of the original RNAs in the sample.

This workflow represents a significant advancement over prior approaches which only separated data points by 2D mass/retention time sigmoidal curves; we now have added a new dimension to aid in data separation 3D mass/retention time/intensity plots (FIG. 9). Using optimal RNA hydrolysis and LC-MS conditions through two control runs to attain the rₐᵥ value, and in silico simulation based on mathematical foundations and reaction kinetics of RNA acid hydrolysis, the fraction of desired ladder fragments were increased; whereas, undesired internal fragments were minimized. The Nested Algorithm, capitalizes on the hydrolysis pattern of RNAs in the sample to derive RNA sequences based on their abundance order in the original RNA sample and the resulting ladder fragments maintained the same relative intensity, i.e., the most abundant RNA generated the most abundant ladder fragments in the sample. Using this 3D approach significantly improved the accuracy and efficiency in sequencing multiple RNAs within a complex mixture.

### The Nested Algorithm.

To identify all the ladder fragments that originated from one parental RNA sequence in the complex LC-MS data of a mixed RNA sample and further de novo read out each RNA sequence, a new method called the Nested Algorithm was developed Named for its matryoshka-doll-like approach, this algorithm effectively leverages the 3D relationship of mass, intensity, and retention time, which are generated simultaneously during LC-MS measurement and are intrinsically related to each RNA or fragment, providing a robust tool to separate data points of ladder fragments into different groups for each RNA and generate its sequence de novo. To achieve this, this algorithm segregates data points of consecutive ladder fragments from the same parent RNA based on the original RNA abundance order into a 3D-mass-intensity-retention time layer. Within each layer, a short RNA sequence can be read by base-calling each nucleotide from mass differences between consecutive ladder fragments. This method efficiently handles MS data separation and separate different RNA sequences on silica, eliminating the need for complex physical sample separation steps.

The Nested Algorithm initiates the 1^{st} layer by identifying the data point (data point A) with a mass lower than the parent RNA and has the highest signal intensity (i.e., the most intense potential ladder fragment). Furthermore, data point A is likely associated with a ladder fragment from the most abundant RNA in the sample. Starting from point A, a base calling identifies the subsequent data point (data point B) with an added mass equivalent to one nucleotide. This point B likely corresponds to a consecutive ladder fragment from the same RNA, recording as an additional nucleotide in the RNA sequence. Once data point B is identified, the process continues iteratively to determine subsequent data points of RNA ladder fragments.

Searching for the next data point follows four criteria: 1) alignment of mass differences with the searching library, encompassing four canonical nucleotides and RNA modifications; 2) fitting appropriate retention time (t_{R}) differences into a 2D t_{R} versus mass sigmoidal curve; 3) selecting the match with highest intensity in cases of multiple matches; and 4) ensuring intensities within the same order of magnitude for consecutive ladder fragments.

This iterative process continues until no further data points can be found to the mass increase direction. Upon reaching this point, the base calling reverts to data point A, searching for a data point likely belonging to the preceding ladder fragment of data point A from the same RNA, containing one less nucleotide. The searching in the mass decreasing direction similarly follows the above four criteria and persists until no more data points can be found. The algorithm persists in processing the remaining data to produce additional arrays of data points belonging to different parent RNAs. Typically, the order of the generated layers of data points aligns with the intensity order of original RNAs in the sample (FIG. 3). The process allows the identification of all the ladder fragments and produces short RNA sequence readouts de novo in each layer. These results can be provided within a few minutes once the deconvoluted MS data files are available, making it ideal for real-time quality control of therapeutic RNA products in the pharmaceutical industry. FIG. 9 illustrates the 3D data separation strategy of the Nested Algorithm. The flowchart of the Nested Algorithm is shown in the Supporting Information (FIG. 8).

### Sequence Assembly to Generate the Full Sequence of RNA.

In complex RNA samples, multiple short RNA sequences are read out from each laver. This occurs because the original search library only contains the nucleotides A, G, C, and U and a few of the many known nucleotide modifications. To construct complete RNA sequences, these short sequences were aligned and assembled by considering nucleotide positions and connections inferred from mass differences involving one or more nucleotide combinations. This is possible because MS sequencing data also carries location information, since the length of an original RNA molecule is approximated by dividing its monoisotopic mass by 320 Da.

Starting with the most intense sequence readout #1, it is aligned with its parallel sequences (arising from partial modifications or metal adducts) characterized by constant mass differences at every nucleotide position corresponding to the same sequence. Bidirectional readout sequences (reading in either 5′→3′ or 3′→5′ directions) are also taken into consideration. The sequence assembly stage also fills the “gaps” caused by modifications not contained in the searching library, including certain rare modifications like 5-carboxymethylaminomethyl-2-geranylthiouridine (cmnm⁵ges²U)⁸. Examples of parallel sequences and gap between sequences are shown in FIG. 10.

The MassSum algorithm⁹ segregates the paired ladder fragments for each RNA sequence and groups short sequences. Prior input of target RNA sequences can be helpful but is not required in the sequence assembly. The final results not only verify the target sequence but also reveal unwanted co-existing RNA sequences.

### Mathematical Foundation and Kinetics of RNA Hydrolysis for Nested Algorithm-Based NGMS-Seq of RNA Mixtures.

### The Kinetics of Acid Hydrolysis of a Phosphor-Ester Bond (P—O Bond).

For an RNA strand with m nucleotides, there are m−1 cleavable phosphodiester bonds (P—O). The acid hydrolysis of a P—O bond is a multi-step reaction. Since the protonation of the phosphate is the rate-determining step¹⁰, P—O hydrolysis follows the second order rate law and the site-specific rate constant for the P—O bond between nucleotide i and i+1 is kᵢ.

$$rate = {{k}_{i} [ P - O ]}_{i} [ {H}_{3} {O}^{+} ] \qquad Equation ( 1 )$$

Because the pH of the acid-hydrolysis is ˜2 and the concentration of RNA molecule is in the μM level, the concentration of [H₃O⁺] is relatively unchanged before and after acid treatment. Thus, the hydrolysis follows the first order rate law at each reaction site:

$${rate}_{i} = {k}_{i}^{′} [ P - O ] , where {k}_{i}^{′} = {k}_{i} [ {H}_{3} {O}^{+} ] \qquad Equation ( 2 )$$

Using the first order integrated rate law:

$$ln ( \frac{{[ P - O ]}_{it}}{{[ P - O ]}_{i 0}} ) = - {k}_{i}^{′} t \qquad Equation ( 3 )$$

where [P—O]ᵢₜ and [P—O]ᵢ₀ are the concentrations of [P—O]ᵢ at time (t) and initial time (0), and t is the hydrolysis time. The ratio [P—O]ᵢₜ/[P—O]ᵢ₀ is the fraction of [P—O] at that specific site i that remains unhydrolyzed. Define rᵢ as the fraction of hydrolyzed P—O at i^{th} site (between nucleotide i and i+1) and combining Arrhenius Equation,

$${k}_{i} = {A}_{i} {e}^{- \frac{{E}_{ai}}{RT}} ,$$

rᵢ can be expressed as:

$$Equation ( 4 )$$

$${r}_{i} = 1 - \frac{{[ P - O ]}_{it}}{{[ P - O ]}_{i 0}} = 1 - {e}^{- {k}_{i}^{′} t} = 1 - {e}^{- {k}_{i} [ {H}_{3} {O}^{+} ] t} = 1 - {e}^{- {A}_{i} {e}^{( - \frac{{E}_{ai}}{RT} )} [ {H}_{3} {O}^{+} ] t}$$

Equation (4) reveals that the magnitude of phosphodiester bond hydrolysis at each site can be determined by experimental conditions including: (1) reaction temperature, (2) formic acid concentration, and (3) hydrolysis time.

### Determination of rₐᵥ

While site-specific fraction of hydrolysis rᵢ values are difficult to determine for all phosphodiester bond, their average value rₐᵥ is readily determined from the intensity ratio of the parent RNA before and after acid-hydrolysis:

$$Equation ( 5 )$$

$$\frac{MS intensity of remaining parent RNA after hydrolysis}{MS intensity of original parent RNA} = \frac{{{C}_{0} ( 1 - {r}_{av} )}^{m - 1}}{{C}_{0}} = {( 1 - {r}_{av} )}^{m - 1}$$

$$Then$$

$${r}_{av} = 1 - \sqrt[m - 1]{\frac{MS intensity of remaining parent RNA after hydrolysis}{MS intensity of original parent RNA}}$$

We have found that the values of rₐᵥ for different RNAs are very close under identical LC-MS conditions as described above and the rₐᵥ can be calculated using the intact RNA intensity ratio obtained from the two control runs.

### Optimal Hydrolysis Time.

With the preliminary data from the two control runs and in silico simulation supported by RNA hydrolysis kinetics, the acid hydrolysis conditions were adjusted to maximize the generation of desired RNA ladder fragments while minimizing undesired internal fragments. The optimal hydrolysis time is determined from information provided in the two control runs. The length of an original RNA molecule is approximated by dividing its monoisotopic mass by 320 Da. The average hydrolysis ratio per site, rₐᵥ, is calculated using Equation (5) (defined in Section 2.2). The theoretical optimal rₐᵥ for a ladder fragment with length k is determined as 1/k, as detailed in (SI). Moreover, the value of rₐᵥ, is directly proportional to hydrolysis time, and a small rₐᵥ (<0.05) allows for a proportional calculation of the theoretical optimal hydrolysis time (FIG. 15). The extensive LC-MS measurements of RNAs revealed that rₐᵥ is approximately 0.004 per minute at 40° C. under the disclosed acid-hydrolysis condition.

Ms Intensity Order of Fragments from Different RNAs Generally Follows the Abundance Order of the Original RNAs in the Sample.

The first order hydrolysis kinetics for RNAs' P—O bonds (Equation (2)) leads to the independent approximation of the probability of forming fragments based on the initial concentration of original RNAs. Therefore, all original RNAs in the sample undergo hydrolysis to a similar extent, and the intensity order of fragments with the same length from different RN As typically aligns with the abundance order of their original RNAs.

The initial relative intensities of three original RNA molecules and the relative intensities of their respective 10 nt 5′ and 3′ ladder fragments, as well as the relative intensity of the 10 nt internal fragments are depicted in FIG. 3B. After acid hydrolysis, the intensity ratio of ladder fragments from different original RNAs roughly follows the intensity ratio of their original RNAs in the control sample (˜9:3:7), whereas, the internal fragments have much lower intensities compared to all the ladder fragments, including the ladder fragments from the least abundant original RNA. Such fragmentation patterns of RNA molecules in a mixture facilitate the precise and fast data separation of ladder fragments based on the intensity, mass, and retention time (FIG. 10).

### Testing the Nested Algorithm-Based NGMS-Seq on a Mixture of Three Small Synthetic RNAs

Small interfering RNAs (siRNAs, 21-27 nts) and microRNAs (miRNAs, 21-23 nts) are key players in regulatory mechanisms and the pathogenesis of diverse diseases, holding valuable therapeutic potential. To mimic therapeutic miRNAs and siRNAs, a small RNA mixture was created by combining three synthetic 20 nt RNAs with a known initial molar ratio of RNA-A:RNA-B:RNA-C=9:3.008:0.604. It was observed that their intensity ratio at the intact level before acid hydrolysis is consistent with the initial molar ratio (FIG. 3B).

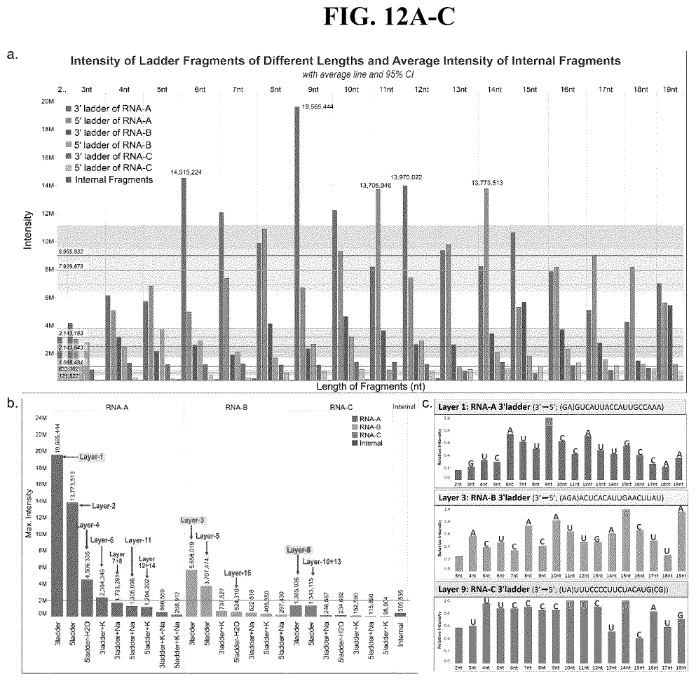

After acid treatment, the resulting 3′ and 5′ ladders fragments from different original RNAs maintained their initial intact abundance ratios. The four criteria (FIG. 8) were used to read out the sequences of the RNAs. Base calling was done within each layer, and the sequences from native form (no adducts) ladder fragments from the three RNAs were determined in the 1^{st}, 2^{nd}, 3^{rd}, 5^{th}, 9^{th}, 10^{th}, and 13^{th} layer, respectively, with 100% accuracy (FIG. 12 and Table 3). The ratio of intensities for the native form of ladder fragments is consistent with that of the intact RNA molecules. Native RNA-A ladders have the highest intensity, followed by RNA-B and RNA-C (FIG. 12A-B).

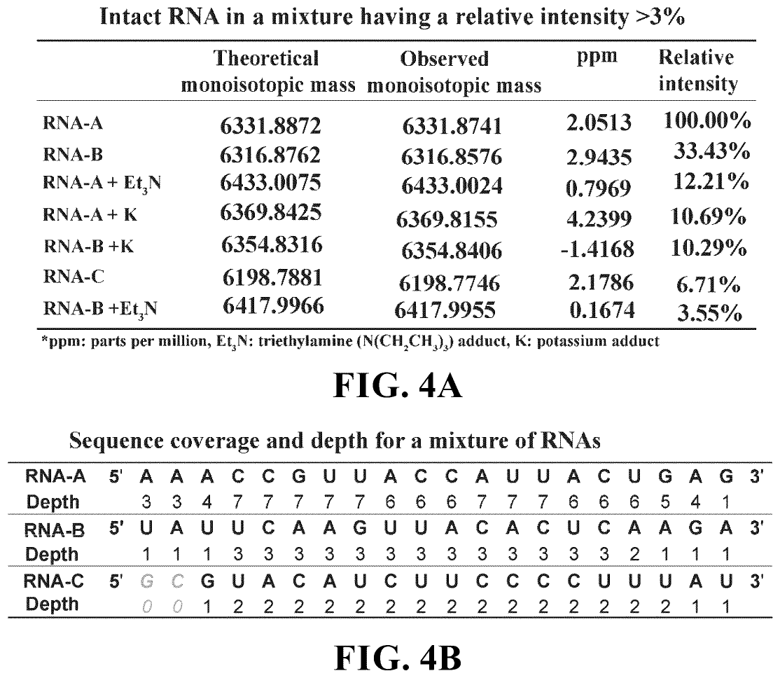

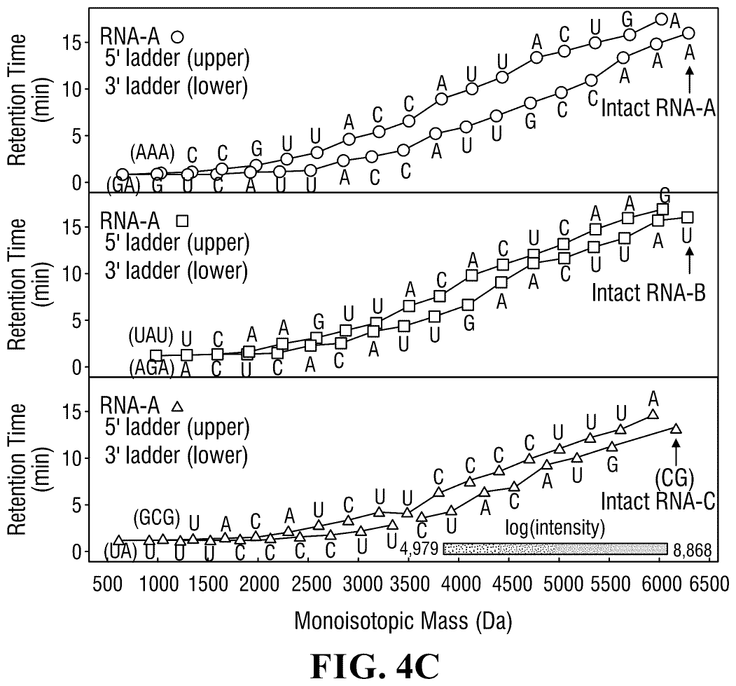

In addition to the native form of RNA molecules, potassium adducts and other forms of RNA molecules were observed (FIG. 4A) at the intact level before acid hydrolysis. Different adduct forms were also observed in the ladder level after acid hydrolysis (FIG. 12B and Table 3) during the layer-by-layer sequencing process. Alongside these native ladder forms (i.e., 3′ and 5′ ladders), additional variations of ladders (dehydrated 5′ ladders, sodium and potassium adduct ladders) also emerged in the top lavers, especially those from the dominant RNA due to their high content (FIG. 12B 2^{nd} and 4^{th} layer for RNA-A). The dehydrated 5′ ladders are intermediates during RNA acid hydrolysis (see FIG. 2B for the schematic of hydrolysis mechanism). The sodium and potassium ions^{11, 12} are possibly introduced during RNA sample preparation and purification. They may potentially impact the efficacy of RNA therapeutics, since these two kinds of adducts are related to numerous physiologic and pathophysiologic processes.

Different forms of 5′ and 3′ ladders can be read out sequentially according to their intensity ranking (FIG. 12B and Table 3), which also helps to increase sequencing depth and minimize errors during the sequence segment assembly stage (FIG. 4B). Due to its highest initial abundance, other forms (different than the native forms of ladders) from RNA-A were also observed and have higher intensities than the ones from less abundant RNAs. RNA-A's sequence was read out more times than RNA-B and RNA-C during the fifteen-layer sequencing. Therefore, RNA-A has a higher sequencing depth (FIG. 4C). On the other hand, RNA-C only had an initial intensity of 6.71% of RNA-A. and its native 3′ and 5′ ladders were only read out in the top 15 layers (at 9^{th} and 10^{th} layers, respectively), and two nucleotides at the 5′ end were not covered due to weak signal intensity. Nevertheless, since the exact mass of different nucleotides is relatively unique, for small ladder fragments containing two or three nucleotides, the combination of nucleotides can still be determined (FIG. 4B).

Nested Algorithm Achieves Full Coverage for CRISPR/Cas9 sgRNA Sequences.

After verifying the nested algorithm-based NGMS-Seq on the three 20 nt synthetic RNA mixture, it was applied to the sequencing of two commercial sgRNA samples (sgRNA1 and sgRNA2). sgRNA1 and sgRNA share similar sequences except that the A at position 7 and the U at position 98 of sgRNA1 were replaced by their 2′-O-methylated counterpart nucleotides designated as an Am or Um. Their sequences differ in the first couple of nucleotides position 1-10 are as follows: sgRNA1 (GUCAGGAUGGCC) (SEQ ID NO: 30) and sgRNA2 (AGUmCGGAmUGGCC) (SEQ ID NO. 11). In the intact sample sgRNA1 without acid treatment, the potassium-adduct sgRNA1 and sgRNA1 have relative intensities of 68.49% and 56.56%, respectively, compared to the most abundant RNA molecule in the sample (identity unknown, mass=32381.27; mass difference to sgRNA1=96.03 Da). On the other hand, in the intact sgRNA2 sample, sgRNA2 and its metal adduct forms are the most abundant.

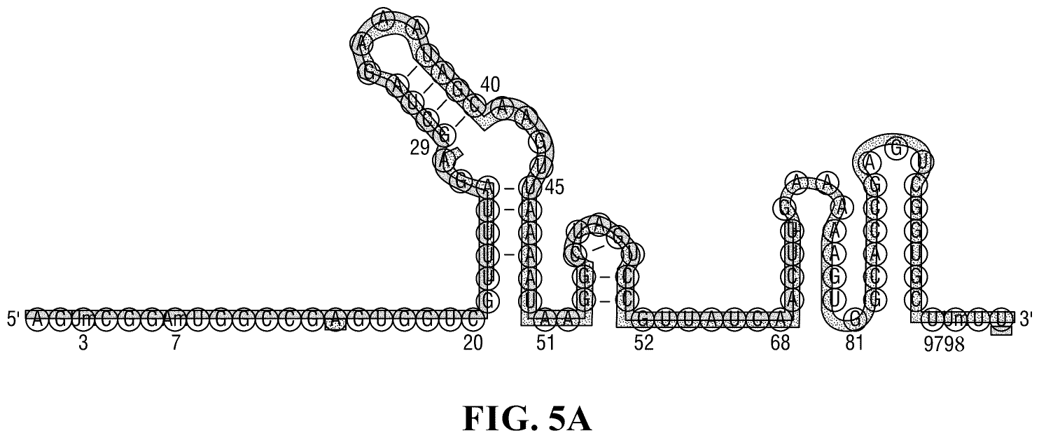

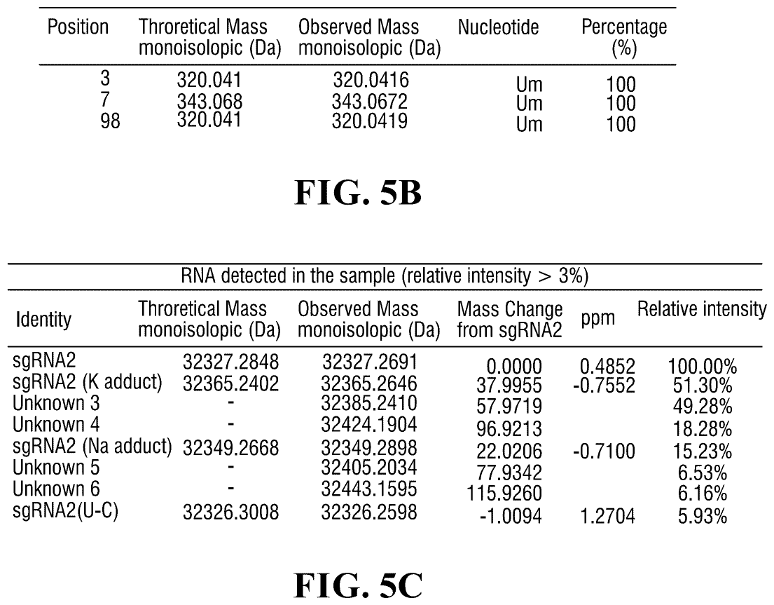

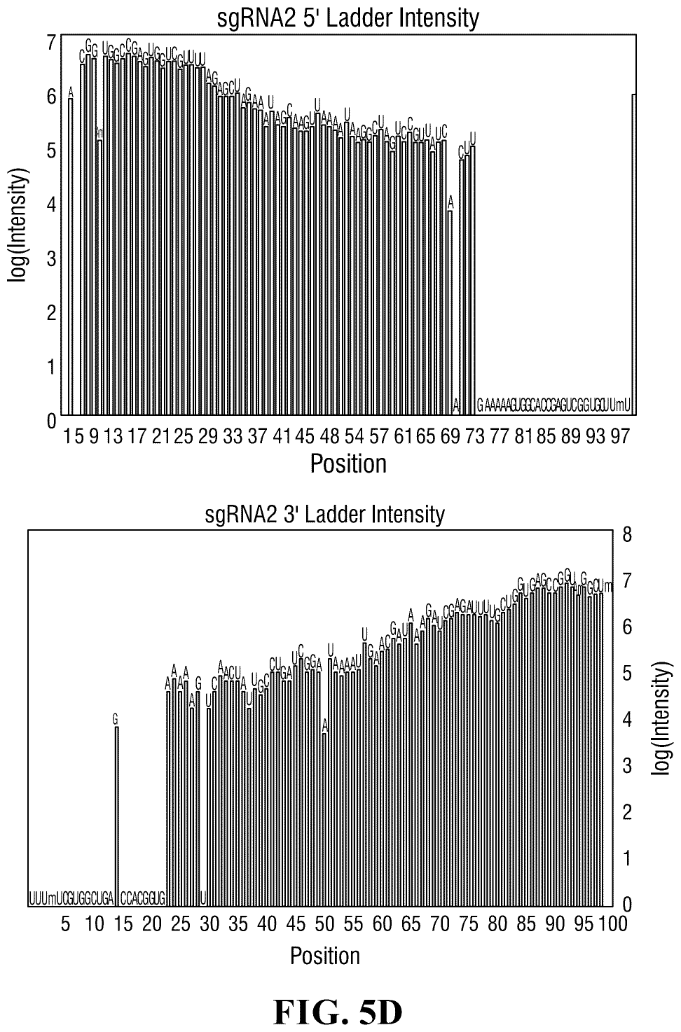

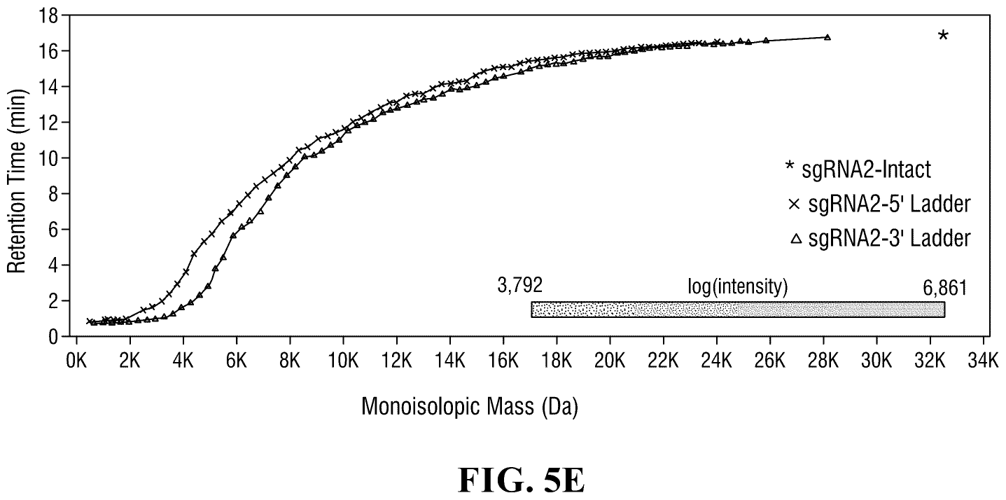

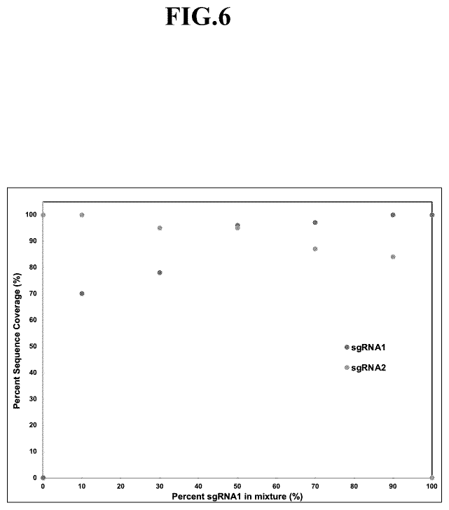

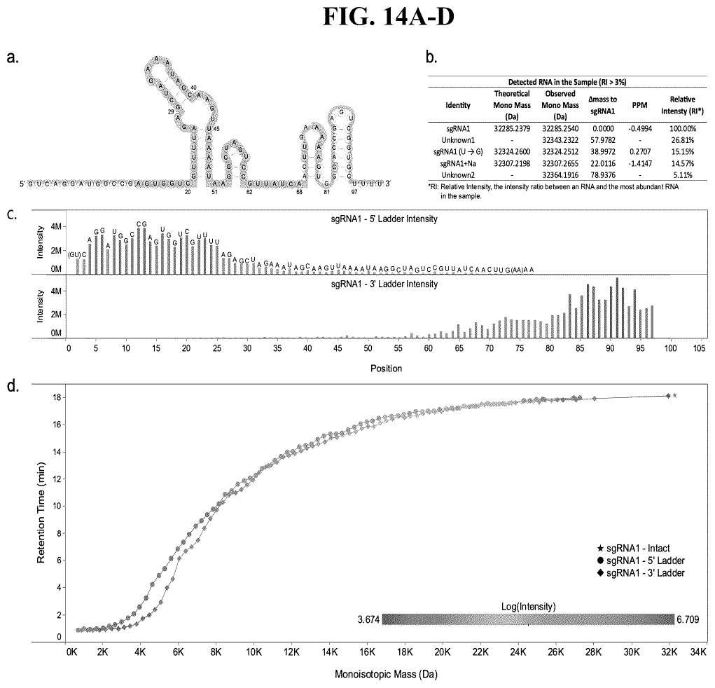

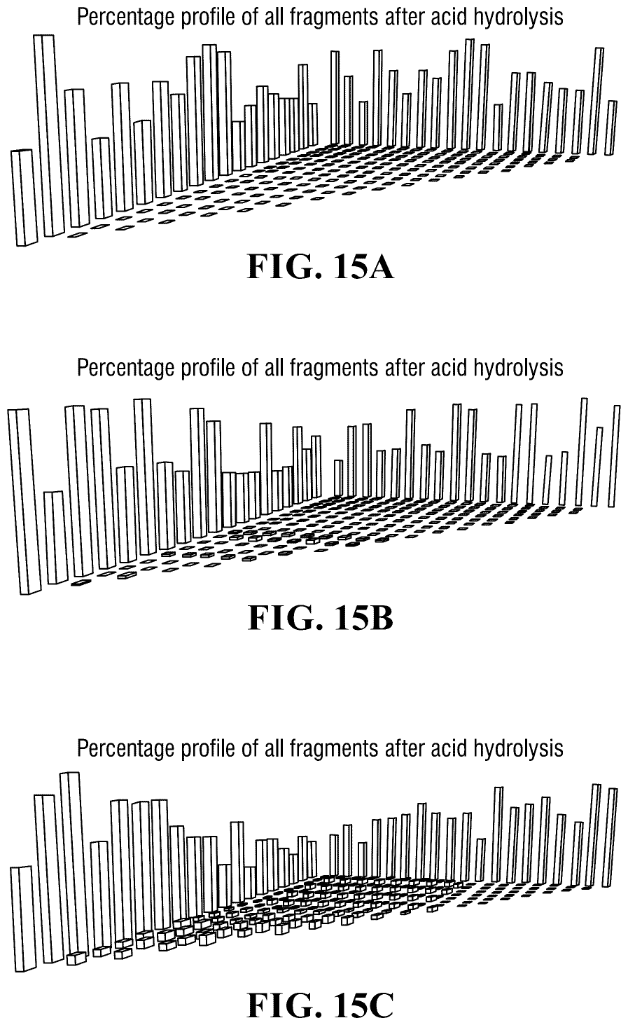

Two individual sgRNA samples were sequenced by systematically identifying and reading out sequences from groups of consecutive ladder fragments in different layers after the analytical runs. To ensure sequencing accuracy, starting points were selected within the mass region of 5000 Da to 6000 Da. This helped correctly pick the 1^{st} most intense ladder fragment with a relative unique sequence. Starting with the most intense ladder fragment candidate in each layer, the nested algorithm accurately grouped related ladder fragments sequentially and read out the sequences from the native and other forms of 3′ and 5′ ladders of sgRNAs with single-nucleotide precision. The top two layers of both sgRNA1 and sgRNA2 samples are their respective native forms of 3′ and 5′ ladders consisting of a series of consecutive ladder fragments. The native forms of 3′ and 5′ ladders overlapped in the middle, achieving full coverage (100%) when sequencing the two sgRNAs (FIG. 14B, FIG. 14C and FIG. 14D, FIG. 5A, FIG. 5D, FIG. 5E, and FIG. 6). For example, the top two layers of the sgRNA1 sample are native 3′ ladders ranging from 3 nts to 72 nts and native 5′ ladders ranging from 2 nts to 68 nts. Moreover, the sequences read out from other forms of ladders further validate the sequences and increase the sequencing depth for the sequences obtained from the native 3′ and 5′ ladders. This significantly improves the sequencing efficiency for RNA molecules approximately 100 nts in length while maintaining high sequencing accuracy, 2′-O-methylations can often be identified based on the fact that methyl groups in this position tend to block the formic acid hydrolysis of the phosphodiester bond. Thus, these fragments do not appear in the MS data, resulting in ladders with a missing fragment. This can be observed in FIG. 5D (left panel), where a missing fragment indicates the presence of a 2′-O-methylation.

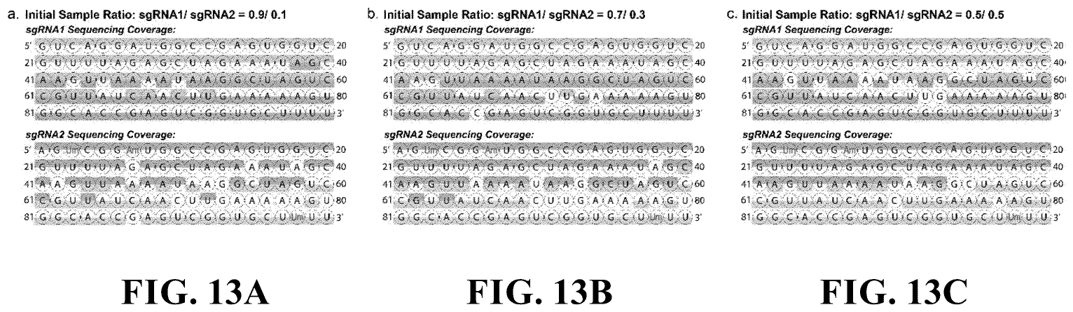

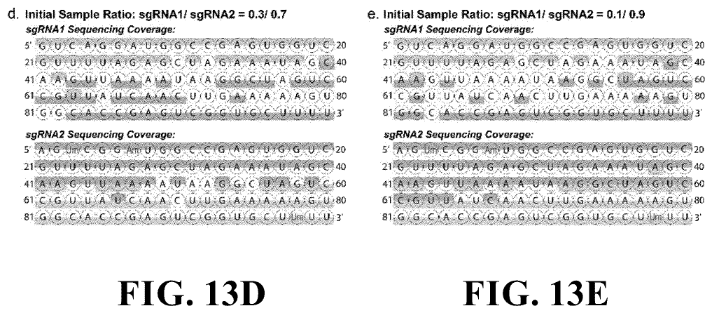

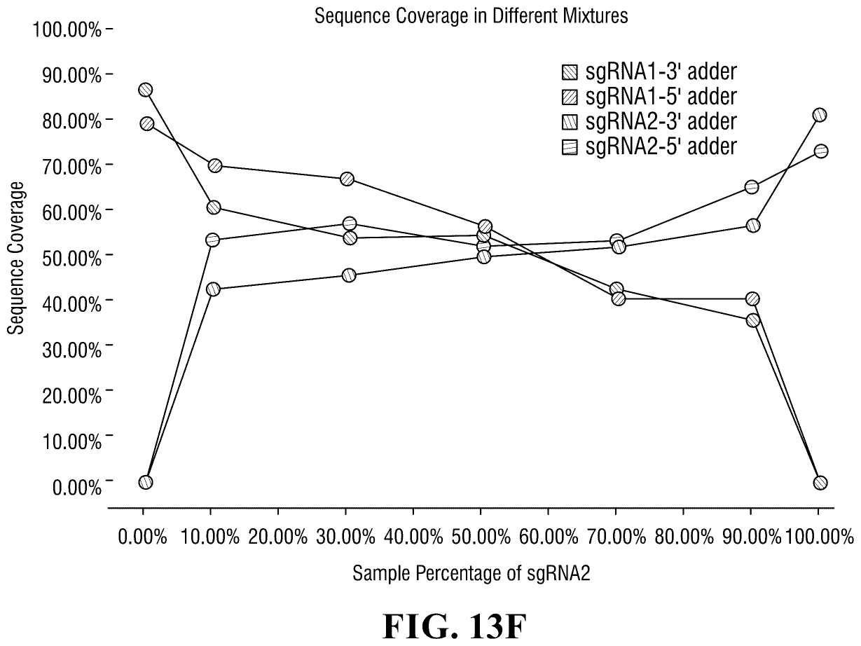

The nested algorithm has the capacity to de novo identify ladder fragments and read out sequences from the mixture of RNAs containing at least two RNA sequences of sgRNA1 and sgRNA2 but with various abundancies. To comprehensively investigate the correlation between sequencing coverage and RNA relative abundance in a mixed sample, five mixtures of sgRNA1 and sgRNA2 were generated with varying known sample ratios. The observed data suggests a positive correlation between sequence coverage and sgRNA abundance (i.e., as the intensity of a sgRNA molecule increases, the resulting sequencing coverage generally also increases; see FIG. 13). Furthermore, a low sgRNA abundancy tends to cause missing 3′ or 5′ ladder fragments. For example, when the initial sample ratio of sgRNA1 and sgRNA2 is 9:1 (FIG. 13), a consecutive 5′ ladder coverage from G1 to U64 and a consecutive 3′ ladder coverage from 100U to 51 A of sgRNA1 is obtained, achieving a full coverage on the entire sgRNA1 sequence. However, due to the low sample abundance of sgRNA2, only a consecutive 5′ ladder coverage from G1 to A26 and a consecutive 3′ ladder coverage from 100U to 88 A was obtained for sgRNA2, covering less than 30 nucleotides at both 5′ and 3 of sgRNA2. This suggests the absence of certain ladder fragments leads to short sequence segments during the layer-by-layer sequencing stage, which emphasizes the importance of sequence assembly in rearranging these short sequence segments and filling “gaps” to obtain the more complete final sequencing results by combining sequence information from other forms of the RNA ladders. On the contrary, when the sgRNA is in a abundancy of >50%, the sequences derived from 3′ and 5′ ladders are relatively complete, providing satisfactory coverage of the sgRNA sequences (see comparison in FIG. 6 and FIG. 13). The sequencing outcomes from the five sgRNA mixtures with different abundances also highlight the effectiveness of the nested algorithm in rapidly and precisely detecting and separating different sgRNA sequences, including both the most abundant and less abundant RNA, within mixed samples, especially considering the high similarity between the sequences of sgRNA1 and sgRNA2.

A Sequencing Scoring System Based on the Distribution of RNA Fragments after Acid Hydrolysis.

To verify the credibility (whether the sequences are truly from original RNAs molecule in the mixture) of the automatic read out sequence segments, a scoring system was developed, which examines the de novo sequences using the positioning of ladder fragments and the length of the sequence segment. Specifically, there are several criteria to evaluate the accuracy of the sequence outcome: an agreement with the references, full coverage of an RNA, identification of parallel sequences, depth, and the intensity and length of the ladder sequences. If the obtained sequences agree with the references, the accuracy score is 100 and if mismatches are found the percentage is deducted. If full coverage of an RNA is achieved, the accuracy score is 100; otherwise, a percentage is deducted based on the number of missing positions. Parallel sequences, derived from data points with fixed mass differences (metal adducts, partial modifications), independently sequenced by the nested algorithm, reduces the probability of random false positive read outs. Some common mass differences between parallel sequences and their explanations are shown in Table 8. Longer segment sequences with higher mass values and MS intensity are more likely to be from a parent RNA in the original mixture. In addition, higher MS intensity contributes to a more reliable outcome, while a decrease in the internal fragment region may affect reliability due to increased base calling false positive rates.

The Sequence Segment Scoring System was developed for quantitative evaluation of sequencing accuracy, especially when a reference sequence is not available, and calculates the probability of false positives at each position based on the base calling pool size and the probability of a random appearance of specific combinations of A, C, G, and U. If a specific A, C, G, and U is not available, it utilizes the maximum false positive from the most common combination to estimate the highest possible false positive value.

For a sequence segment read from the Nesting Algorithm covering positions #h to #k, the corresponding data points to the RNA's ladder fragments at #h−1 to #k must be assigned. The false positive for the existence of all k−h+1 data points due to randomness is calculated as the product of false positive values at all k−h+1 positions. The accuracy score of the sequence is then defined as 100 times (1 minus the product of these false positives). A score of 95 means there is 5% chance this segment sequence is the result of the random distribution of the data.

As an illustrative example, if the base calling pool size is 90, a sequence covering positions #5-14 has a score of 54, and a sequence covering positions #15-24 has a score of 99. If the base calling pool size is 200, then the corresponding scores are 6 and 69, respectively. Larger base calling pool sizes increase data complexity and decrease the score. Higher positions in the sequences and longer sequences result in higher scores. This scoring system was applied to the 3 synthetic RNAs, since all 15 sequences have reference sequence support, and their accuracy scores are 100.

Impurities in the Synthetic sgRNA Sequences Identified Using the Nested Algorithm

In the sgRNA1 and sgRNA2 samples before acid hydrolysis, RNA impurities (intact level) likely introduced as by-products during chemical synthesis were detected (FIG. 5B and FIG. 14B).

The sgRNA2 sample has a higher purity than sgRNA1, although some impurities are still observed (FIG. 5B). Additionally, in the control sample of sgRNA1, intense peaks from partially degraded sgRNA with masses around 10,800 Da (about 33 nts) were observed (FIG. 14B).

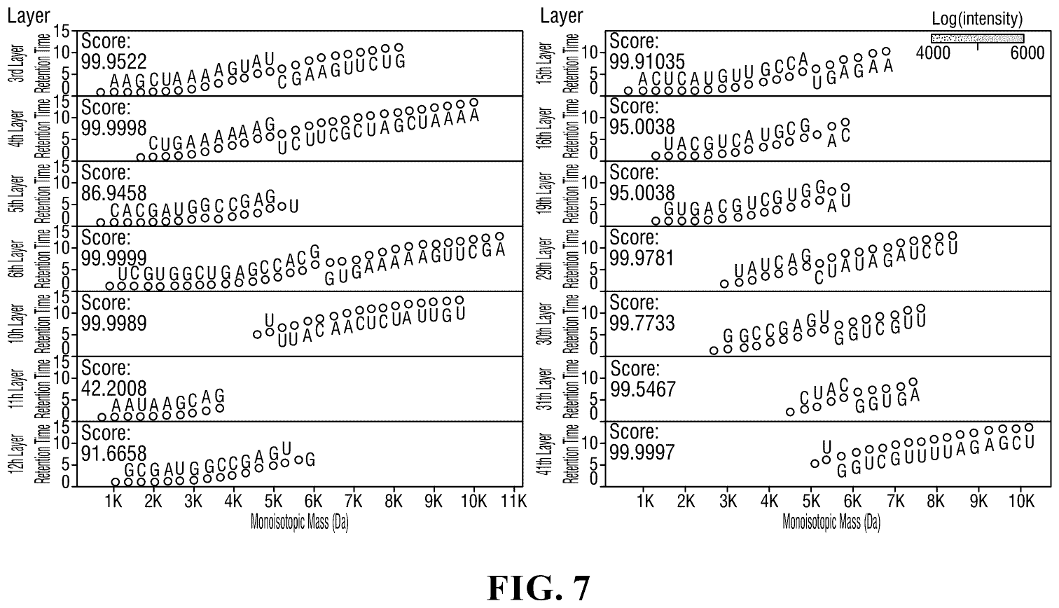

The ladder fragments and sequences associated with some potential impurities (ladder level) were also extracted, considering the impurities observed at the intact level before acid hydrolysis. To assess the credibility of these sequences (some of which are unrelated to the target RNAs. i.e., undesired impurities), scores were assigned using the proposed scoring system. FIG. 7 illustrates the 14 sequences obtained from the top 50 layers during the layer-by-layer sequencing by the nested algorithm. The sequences obtained from the 3rd, 4th, 10th, 11th, 15th, 16th, 19th, and 29th layers are proven unrelated sequences and unrelated ladder fragments to the sgRNA1 after comparing with the theoretical sequences and theoretical mass of ladder fragments. Besides using the smoothness of the (R-monoisotopic sigmoid curve and intensity similarities between consecutive ladder fragments to help validate the ladder fragment belongings, a score of larger than 90 (based on probability and ladder positioning) can also help to assess whether one should group these ladder fragments together. Specifically, the sequence based on the ladder fragments of length from 2 nts to 11 nts in the 11th layer has a score of 42.2008, exhibiting lower reliability compared to the ones with a score larger than 90, such as the sequence from the 3rd layer has a score of 99.9522 (ladder fragments of length from 2 nts to 25 nts). Nevertheless, the nested algorithm coupled with the scoring system provides us with a fast and efficient RNA sequencing framework to de novo sequence RNAs from mixtures, assess and assemble sequence segments, as well as quantitatively examine the impurities in the sample.

Here it is shown that 20 nt mixtures and long sgRNAs (100 nt) and their mixtures can be sequenced using the Nested Algorithm; however, this method can also be potentially applied to cellular RNAs. Since the Nested Algorithm needs good data, balancing several factors and optimizing the sequencing strategy based on the specific characteristics of the RNA sample is crucial for obtaining reliable and accurate results. Higher signal strengths contribute to a reliable and accurate base calling, and this can be achieved by using an optimal amount of sample in the acid-hydrolysis. Also having less overlapping MS signals is advantageous; this requires optimizing of RNA acid hydrolysis and LC-MS running conditions. In addition, managing complexity is essential for accurate base calling and trustworthy sequencing outcomes, since a greater complexity may require careful consideration of nucleotide modifications in the library levels, filtering criteria, and sequence assembly strategies. Having a higher sequence cover improves confidence in the identified sequences and reduces the risk of false positives; a higher sequence depth increases the reliability of the sequences obtained. This NGMS-Seq provides a much-needed tool for sequencing therapeutic RNAs and their modifications.

The NGMS-Seq method measures intact sgRNA masses up to ˜32 kDa without a T1 digestion, bypassing traditional MS/MS techniques dependent on pre-established sequences and enzymatic digestion. This method also measures and quantifies acid hydrolyzed RNA ladder fragments, enabling the sequencing of full-length sgRNAs and their impurities. Thus. NGMS-seq provides a comprehensive quantitative profile of each RNA and its modifications in a sample, bridging a key gap in current methods and holds great promise in drug development.

Since this method is able to distinguish ladders generated from intact RNAs in a mixture, using multi-dimensional data information, the laver-by-layer method is being extended to sequence complex cellular RNA and to investigate biologically significant questions, such as changes in modifications or expression levels after different treatments. The unique design, high accuracy, strong resolving power and ease of use makes NGMS-Seq a promising method for both pharmacological and biological studies.

The documents listed below and referenced herein are incorporated herein by reference in their entireties, except for any statements contradictory to the express disclosure herein, subject matter disclaimers or disavowals, and except to the extent that the incorporated material is inconsistent with the express disclosure herein, in which case the language in this disclosure controls. Incorporation by reference of the following shall not be considered an admission by the applicant that the incorporated materials are prior art to the present disclosure, nor shall any document be considered material to patentability of the present disclosure.

### REFERENCE

1. Chehelgerdi, M. & Chehelgerdi, M. The use of RNA-based treatments in the field of cancer immunotherapy. Mol Cancer 22, 106 (2023).
2. Boudewijns, S. et al. Autologous monocyte-derived DC vaccination combined with cisplatin in stage III and IV melanoma patients: a prospective, randomized phase 2 trial. Cancer Immunol Immunother 69, 477-488 (2020).
3. Liu. Z. et al. Recent advances and applications of CRISPR-Cas9 in cancer immunotherapy Mol Cancer 22, 35 (2023).
4. Goyon, A., Scott, B., Crittenden, C. M. & Zhang, K. Analysis of pharmaceutical drug oligomers by selective comprehensive two-dimensional liquid chromatography-high resolution mass spectrometry. J Pharm Biomed Anal 208, 114466 (2022).
5. Goyon, A. et al. Full Sequencing of CRISPR/Cas9 Single Guide RNA (sgRNA) via Parallel Ribonuclease Digestions and Hydrophilic Interaction Liquid Chromatography-High-Resolution Mass Spectrometry Analysis. Anal Chem 93, 14792-14801 (2021).
6. Wetzel, C. & Limbach, P. A. Mass spectrometry of modified RNAs: recent developments. Analyst 141, 16-23 (2016).
7. Zhang, Ning et al. 2D-HELS MS Seq: A General LC-MS-Based Method for Direct and de novo Sequencing of RNA Mixtures with Different Nucleotide Modifications. Journal of visualized experiments. JoVE, 161 10.3791/61281 (2020).
8. Zheng. Chenkang et al. Diverse Mechanisms of Sulfur Decoration in Bacterial tRNA and Their Cellular Functions. Biomolecules vol. 7, 133. (2017).
9. Shenglong Zhang, Xiaohong Yuan, Yue Su et al. MLC-Seq: de novo Sequencing of Full-Length IRNAs and Quantitative Mapping of Multiple RNA Modifications, 9 Dec. 2021, PREPRINT (Version 1) available at Research Square
10. Bjorkbom, A. et al. Bidirectional Direct Sequencing of Noncanonical RNA by Two-Dimensional Analysis of Mass Chromatograms. J Am Chem Soc 137, 14430-14438 (2015).
11. Pohl, H. R., Wheeler, J. S., Murray, H. E. Sodium and Potassium in Health and Disease. In: Sigel, A., Sigel, H., Sigel, R. (eds) Interrelations between Essential Metal Ions and Human Diseases. Metal Ions in Life Sciences, vol 13. Springer, Dordrecht. (2013).
12. Birdsall, Robert E et al. Reduction of metal adducts m oligonucleotide mass spectra in ion-pair reversed-phase chromatography/mass spectrometry analysis. Rapid communications in mass spectrometry: RCM vol. 30.14:1667-1679 (2016).
13. Yin, H., Song, C Q., Dorkin, J. et al. Therapeutic genome editing by combined viral and non-viral delivery of CRISPR system components in vivo. Nat Biotechnol 34, 328-333 (2016)
14. Miller, Jason B et al. Non-Viral CRISPR/Cas Gene Editing In Vitro and In Vivo Enabled by Synthetic Nanoparticle Co-Delivery of Cas9 mRNA and sgRNA. Angewandte Chemie (International ed. in English) vol. 56, 4:1059-1063 (2017).
15. Wang, Dan et al. CRISPR-Based Therapeutic Genome Editing: Strategies and In Vivo Delivery by AAV Vectors. Cell vol. 181, 1:136-150 (2020).
16. Gee, P., Lung, M. S. Y., Okuzaki, Y. et al. Extracellular nanovesicles for packaging of CRISPR-Cas9 protein and sgRNA to induce therapeutic exon skipping. Nat Commun 11, 1334 (2020).

TABLE 1
Calculated fractions from acid-hydrolysis at different time and
their comparison against previous splitting-pooling approach.

| | Hydrolysis time = 3.25 | | | Hydrolysis time = 5 | | | | | |
|---|---|---|---|---|---|---|---|---|---|
| | min, rₐᵥ = 0.013 | | | min, rₐᵥ = 0.02 | | | Hydrolysis time = 1, | | |
| | | | vs | | | vs | 2, 5 and 10 min, pooled | | |
| | | L/I | pooled | | L/I | pooled | | | L/I |
| length | Ladder | ratio | ratio | Ladder | ratio | ratio | Ladder | internal | ratio |
| 10 | 0.011556 | 77 | 0.83 | 0.016675 | 50 | 1.20 | 0.013919 | 0.000379 | 37 |
| 20 | 0.010138 | 77 | 0.95 | 0.013625 | 50 | 1.28 | 0.010654 | 0.00027 | 39 |
| 30 | 0.008895 | 77 | 1.07 | 0.011132 | 50 | 1.34 | 0.008319 | 0.000194 | 43 |
| 40 | 0.007804 | 77 | 1.18 | 0.009096 | 50 | 1.37 | 0.006626 | 0.000142 | 47 |
| 50 | 0.006847 | 77 | 1.27 | 0.007432 | 50 | 1.38 | 0.005382 | 0.000105 | 51 |
| 60 | 0.006007 | 77 | 1.35 | 0.006073 | 50 | 1.36 | 0.004452 | 7.95E−05 | 56 |
| 70 | 0.00527 | 77 | 1.41 | 0.004962 | 50 | 1.32 | 0.003746 | 6.1E−05 | 61 |
| 80 | 0.004624 | 77 | 1.44 | 0.004054 | 50 | 1.27 | 0.0032 | 4.76E−05 | 67 |
| 90 | 0.004057 | 77 | 1.46 | 0.003312 | 50 | 1.20 | 0.002771 | 3.78E−05 | 73 |
| 100 | 0.003559 | 77 | 1.47 | 0.002707 | 50 | 1.11 | 0.002428 | 3.05E−05 | 80 |

Data shows that hydrolysis outcomes at 5 minutes outperform the splitting-pooling approach by 11-38% for all ladder fragments. Hydrolysis at 3.25 minutes (calculated optimal time for 77 nt RNA) have the best fractions for ladders with length greater than 60.

TABLE 2
Intensity ratio of intact and ladder fragments from RNA-A,
RNA-B, and RNA-C in the control sample (no acid treatment)
and in the acid hydrolyzed sample (with acid treatment).

| | |
|---|---|
| | Intact/Original Strand Intensity Ratios |
| Control, original RNAs | RNA-A:RNA-B:RNA-C = 9:3.008:0.604 |
| Acid hydrolysis sample | RNA-A:RNA-B:RNA-C = 9:2.620:0.371 |
| | Average Ladder Intensity Ratios in |
| | Hydrolyzed Sample |
| 3′ Ladder Fragme nts | RNA-A:RNA-B:RNA-C = 9:3.148:1.090 |
| 5′ Ladder Fragme nts | RNA-A:RNA-B:RNA-C = 9:2.433:0.719 |

TABLE 3
Fifteen-layer NGMS-Seq results of the mixture of
three synthetic RNAs (RNA-A, RNA-B, and RNA-C).

| Monoisotopic | Retention | | Layer | |
|---|---|---|---|---|
| Mass | Time | Intensity | number | Layer Identity |
| 612.1421782 | 1.00617129 | 3159000 | layer 1 | 3′ ladder fragments of |
| 957.1885 | 1.00617129 | 4162546 | layer 1 | RNA-A (2 nts-19 nts + |
| 1263.212874 | 1.00617129 | 6144400 | layer 1 | intact) (1st strand of this |
| 1568.253614 | 1.00617129 | 5695807 | layer 1 | layer: 9 nt 3′ ladder) |
| 1897.304337 | 1.16559662 | 14515224 | layer 1 | |
| 2203.330103 | 1.24184666 | 12050658 | layer 1 | |
| 2509.354425 | 1.39416464 | 9842499 | layer 1 | |
| 2838.405228 | 2.40277429 | 19565444 | layer 1 | |
| 3143.446625 | 2.79199317 | 12184746 | layer 1 | |
| 3448.486583 | 3.48624602 | 8168771 | layer 1 | |
| 3777.537552 | 5.3472757 | 13970022 | layer 1 | |
| 4083.564462 | 6.04902711 | 9319186 | layer 1 | |
| 4389.587417 | 7.13178641 | 8196152 | layer 1 | |
| 4734.632893 | 8.52750055 | 10630510 | layer 1 | |
| 5039.674668 | 9.53432048 | 7823986 | layer 1 | |
| 5344.710836 | 10.7704855 | 5093575 | layer 1 | |
| 5673.774312 | 13.2548414 | 4244413 | layer 1 | |
| 6002.814973 | 14.9567439 | 6978041 | layer 1 | |
| 6331.866884 | 15.9668029 | 7.37E+08 | layer 1 | |
| 1005.165244 | 1.16559662 | 3017469 | layer 2 | 5′ ladder fragments of |
| 1310.205423 | 1.24184666 | 5049220 | layer 2 | RNA-A (3 nt-19 nt) (1st |
| 1615.246194 | 1.47057248 | 6805604 | layer 2 | strand of this layer: 14 nt 5′ |
| 1960.292944 | 1.86028153 | 4966276 | layer 2 | ladder) |
| 2266.317802 | 2.47913207 | 7339938 | layer 2 | |
| 2572.342113 | 3.18111905 | 10825692 | layer 2 | |
| 2901.39415 | 4.64573521 | 6685390 | layer 2 | |
| 3206.435227 | 5.49969428 | 9297697 | layer 2 | |
| 3511.473928 | 6.66681935 | 13706946 | layer 2 | |
| 3840.523515 | 8.99270439 | 7385913 | layer 2 | |
| 4146.549359 | 9.99963588 | 9751209 | layer 2 | |
| 4452.574607 | 11.1594611 | 13773513 | layer 2 | |
| 4781.633128 | 13.2548414 | 5324409 | layer 2 | |
| 5086.665413 | 14.0264502 | 8126367 | layer 2 | |
| 5392.688795 | 14.8805006 | 9012612 | layer 2 | |
| 5737.716284 | 15.7327353 | 8120067 | layer 2 | |
| 6066.791249 | 17.358985 | 5619526 | layer 2 | |
| 941.1936 | 1.00617129 | 1390311 | layer 3 | 3′ ladder fragments of |
| 1270.245627 | 1.08954221 | 3164170 | layer 3 | RNA-B (3 nt-19 nt + intact) |
| 1575.286183 | 1.08954221 | 2157554 | layer 3 | (1st strand of this layer: |
| 1881.310699 | 1.16559662 | 2583818 | layer 3 | 15 nt 3′ ladder) |
| 2186.352404 | 1.31794893 | 1880528 | layer 3 | |
| 2515.403405 | 2.01321027 | 4113504 | layer 3 | |
| 2820.443043 | 2.32645456 | 2304787 | layer 3 | |
| 3149.494294 | 3.63878251 | 4621788 | layer 3 | |
| 3455.519317 | 4.18805015 | 3597312 | layer 3 | |
| 3761.543162 | 5.1947902 | 2637902 | layer 3 | |
| 4106.589118 | 6.50677346 | 2607084 | layer 3 | |
| 4435.64127 | 8.84012249 | 3394657 | layer 3 | |
| 4764.693062 | 10.8544148 | 5658019 | layer 3 | |
| 5069.735554 | 11.3885268 | 3694855 | layer 3 | |
| 5375.763965 | 12.4763086 | 2745132 | layer 3 | |
| 5681.788267 | 13.5607788 | 1476559 | layer 3 | |
| 6010.822308 | 15.5737242 | 5406138 | layer 3 | |
| 6316.848069 | 15.8083391 | 2.15E+08 | layer 3 | |
| 987.1548982 | 1.08954221 | 1238440 | layer 4 | Dehydrated 5′ ladder |
| 1292.195302 | 1.08954221 | 2019832 | layer 4 | fragments of RNA-A (3 nt- |
| 1597.235939 | 1.24184666 | 2771468 | layer 4 | 19 nt) (1st strand of this |
| 1942.281797 | 1.47057248 | 2518384 | layer 4 | layer: 11 nt dehydrated 5′ |
| 2248.307665 | 1.86028153 | 2830333 | layer 4 | ladder) |
| 2554.33105 | 2.40277429 | 3227940 | layer 4 | |
| 2883.380662 | 3.56248457 | 1802001 | layer 4 | |
| 3188.418318 | 4.34063615 | 2693299 | layer 4 | |
| 3493.462825 | 5.2710376 | 4506335 | layer 4 | |
| 3822.50125 | 7.28433803 | 1954308 | layer 4 | |
| 4128.536096 | 8.44362825 | 2745217 | layer 4 | |
| 4434.564554 | 9.68689464 | 2601860 | layer 4 | |
| 4763.606075 | 11.7018604 | 839226.3 | layer 4 | |
| 5068.645326 | 12.7056089 | 1316012 | layer 4 | |
| 5374.680266 | 13.7135549 | 1367800 | layer 4 | |
| 5719.725586 | 14.8043241 | 1097578 | layer 4 | |
| 6048.77994 | 15.5737242 | 1199594 | layer 4 | |
| 959.1097 | 1.00617129 | 2764495 | layer 5 | 5′ ladder fragments of |
| 1265.135107 | 1.08954221 | 2469619 | layer 5 | RNA-B (3 nt-19 nt) (1st |
| 1570.175291 | 1.16559662 | 3707474 | layer 5 | strand of this layer: 5 nt 5′ |
| 1899.22848 | 1.47057248 | 2887127 | layer 5 | ladder) |
| 2228.280824 | 2.25017596 | 2097269 | layer 5 | |
| 2573.329229 | 2.86834258 | 1691310 | layer 5 | |
| 2879.350968 | 3.63878251 | 2680896 | layer 5 | |
| 3185.37578 | 4.49319476 | 3197172 | layer 5 | |
| 3514.431382 | 6.27809219 | 825076.6 | layer 5 | |
| 3819.468369 | 7.43677619 | 2916527 | layer 5 | |
| 4148.529368 | 9.61061228 | 1005608 | layer 5 | |
| 4453.562776 | 10.6178022 | 2056155 | layer 5 | |
| 4759.585349 | 11.7018604 | 1803859 | layer 5 | |
| 5064.626623 | 12.789502 | 2305445 | layer 5 | |
| 5393.68272 | 14.5679751 | 1561307 | layer 5 | |
| 5722.704992 | 15.7327353 | 1238407 | layer 5 | |
| 6067.781708 | 16.6568462 | 1234184 | layer 5 | |
| 1301.159358 | 1.00617129 | 469418.8 | layer 6 | 3′ ladder strand + K of |
| 1606.200041 | 1.00617129 | 390335.6 | layer 6 | RNA-A (4 nt-19 nt and |
| 1935.250496 | 1.16559662 | 1794076 | layer 6 | intact + K) |
| 2241.276288 | 1.24184666 | 1435292 | layer 6 | (1st strand of this layer: 9 nt |
| 2547.300627 | 1.39416464 | 1131751 | layer 6 | 3′ ladder + K) |
| 2876.354787 | 2.40277429 | 2364349 | layer 6 | |
| 3181.393897 | 2.79199317 | 1669053 | layer 6 | |
| 3486.43216 | 3.48624602 | 900381.7 | layer 6 | |
| 3815.484886 | 5.3472757 | 1462158 | layer 6 | |
| 4121.511129 | 6.04902711 | 1025340 | layer 6 | |
| 4427.535575 | 7.13178641 | 861557.8 | layer 6 | |
| 4772.578854 | 8.52750055 | 1011764 | layer 6 | |
| 5077.619461 | 9.53432048 | 753522.3 | layer 6 | |
| 5382.658752 | 10.7704855 | 520433.9 | layer 6 | |
| 5711.719036 | 13.2548414 | 404705.2 | layer 6 | |
| 6040.762059 | 14.9567439 | 655106.6 | layer 6 | |
| 6369.805426 | 15.9668029 | 73859929 | layer 6 | |
| 979.170126 | 1.00617129 | 1096876 | layer 7 | 3′ ladder strand + Na of |
| 1285.194798 | 1.00617129 | 1191442 | layer 7 | RNA-A (3 nt-9 nt) (1st |
| 1590.234459 | 1.00617129 | 679656.8 | layer 7 | strand of this layer: 9 nt 3′ |
| 1919.284872 | 1.16559662 | 1069621 | layer 7 | ladder + Na) |
| 2225.305831 | 1.24184666 | 1292053 | layer 7 | |
| 2531.330422 | 1.39416464 | 885918.1 | layer 7 | |
| 2860.38462 | 2.40277429 | 1733281 | layer 7 | |
| 3470.464672 | 3.48624602 | 722566 | layer 8 | 3′ ladder strand + Na of |
| 3799.511569 | 5.2710376 | 1616994 | layer 8 | RNA-A (11 nt-19 nt and |
| 4105.542915 | 6.04902711 | 832350.1 | layer 8 | Intact + Na) (1st strand of |
| 4411.566111 | 7.13178641 | 724943.1 | layer 8 | this layer: 12 nt 3′ |
| 4756.612875 | 8.52750055 | 878014.5 | layer 8 | ladder + Na) |
| 5061.655768 | 9.53432048 | 727391.1 | layer 8 | |
| 5366.690205 | 10.7704855 | 500672.8 | layer 8 | |
| 5695.749721 | 13.2548414 | 409348.4 | layer 8 | |
| 6024.790372 | 14.9567439 | 601944.9 | layer 8 | |
| 6353.841892 | 16.0424035 | 33232989 | layer 8 | |
| 573.1201738 | 1.00617129 | 791935.3 | layer 9 | 3′ ladder fragments of |
| 879.1441 | 0.93004242 | 824719.1 | layer 9 | RNA-C (2 nt-18 nt) (1st |
| 1185.169144 | 1.00617129 | 1320729 | layer 9 | strand of this layer: 14 nt 3′ |
| 1491.193373 | 1.00617129 | 1208790 | layer 9 | ladder) |
| 1796.233966 | 1.08954221 | 1207305 | layer 9 | |
| 2101.273737 | 1.16559662 | 1246604 | layer 9 | |
| 2406.314816 | 1.31794893 | 1171474 | layer 9 | |
| 2711.356962 | 1.5469707 | 1169774 | layer 9 | |
| 3017.381046 | 2.01321027 | 1384127 | layer 9 | |
| 3323.405967 | 2.63179067 | 1380755 | layer 9 | |
| 3628.443465 | 3.41000742 | 1243553 | layer 9 | |
| 3934.465525 | 4.11176989 | 703652.3 | layer 9 | |
| 4263.522013 | 6.20185335 | 1385036 | layer 9 | |
| 4568.562705 | 6.66681935 | 554027.9 | layer 9 | |
| 4897.611075 | 8.91641193 | 1142279 | layer 9 | |
| 5203.636324 | 9.68689464 | 798038.5 | layer 9 | |
| 5548.681249 | 10.8544148 | 970519.9 | layer 9 | |
| 3810.46762 | 6.12549365 | 679317.1 | layer 10 | 5′ ladder fragments of |
| 4115.50425 | 7.20804752 | 871628.8 | layer 10 | RNA-C (12 nt-19 nt) (1st |
| 4420.546452 | 8.36741589 | 902150.6 | layer 10 | strand of this layer: 16 nt 5′ |
| 4725.586663 | 9.53432048 | 1019294 | layer 10 | ladder) Due to 11 nt |
| 5031.612305 | 10.6178022 | 1343115 | layer 10 | 5′ladder missing, there are |
| 5337.636906 | 11.7018604 | 1136022 | layer 10 | 5 wrong ones but the |
| 5643.663016 | 12.7056089 | 890912.7 | layer 10 | intensity dropped |
| 5972.708089 | 14.4154046 | 396857.5 | layer 10 | dramatically from 265->676. |
| 698.0954935 | 1.08954221 | 187426.7 | layer 11 | 5′ ladder strand + Na of |
| 1027.146571 | 1.08954221 | 196982.3 | layer 11 | RNA-A (2 nt-19 nt) (1st |
| 1332.185348 | 1.24184666 | 316938.9 | layer 11 | strand of this layer: 14 nt 5′ |
| 1637.225743 | 1.39416464 | 398662.2 | layer 11 | ladder + Na) |
| 1982.271779 | 1.86028153 | 321732.3 | layer 11 | |
| 2288.295729 | 2.47913207 | 511709.9 | layer 11 | |
| 2594.320033 | 3.18111905 | 826074.8 | layer 11 | |
| 2923.372772 | 4.64573521 | 578660.6 | layer 11 | |
| 3228.413086 | 5.57591804 | 777067.3 | layer 11 | |
| 3533.453199 | 6.66681935 | 1132637 | layer 11 | |
| 3862.501266 | 8.99270439 | 604948.1 | layer 11 | |
| 4168.526484 | 9.99963588 | 914247.7 | layer 11 | |
| 4474.551494 | 11.1594611 | 1305096 | layer 11 | |
| 4803.609918 | 13.2548414 | 431839.9 | layer 11 | |
| 5108.642874 | 14.0264502 | 743095.9 | layer 11 | |
| 5414.666288 | 14.8805006 | 937839.7 | layer 11 | |
| 5759.696312 | 15.7327353 | 450044.6 | layer 11 | |
| 6088.77414 | 17.358985 | 580520.7 | layer 11 | |
| 1043.112003 | 1.16559662 | 269197 | layer 12 | 5′ ladder strand + K of |
| 1348.151845 | 1.24184666 | 421045.6 | layer 12 | RNA-A (3 nt-14 nt) (1st |
| 1653.192896 | 1.47057248 | 583889.4 | layer 12 | strand of this layer: 14 nt 5′ |
| 1998.239446 | 1.86028153 | 410229.8 | layer 12 | ladder + K) |
| 2304.264669 | 2.47913207 | 666219.8 | layer 12 | |
| 2610.288266 | 3.18111905 | 1058912 | layer 12 | |
| 2939.342636 | 4.64573521 | 548506.6 | layer 12 | |
| 3244.38273 | 5.49969428 | 774029.1 | layer 12 | |
| 3549.418302 | 6.66681935 | 1153920 | layer 12 | |
| 3878.469587 | 8.99270439 | 506519.3 | layer 12 | |
| 4184.494547 | 9.99963588 | 810969.1 | layer 12 | |
| 4490.521105 | 11.1594611 | 1204202 | layer 12 | |
| 1013.143278 | 1.08954221 | 95208.72 | layer 13 | 5′ ladder fragments of |
| 1319.168601 | 1.08954221 | 276952.7 | layer 13 | RNA-C (3 nt-10 nt) (1st |
| 1648.219451 | 1.24184666 | 166357.1 | layer 13 | strand of this layer: 10 nt 5′ |
| 1953.260185 | 1.31794893 | 410662.3 | layer 13 | ladder) |
| 2282.31182 | 1.93676979 | 271060.8 | layer 13 | |
| 2588.339018 | 2.47913207 | 597517.3 | layer 13 | |
| 2893.378725 | 3.18111905 | 715870.9 | layer 13 | |
| 3199.402531 | 4.02783816 | 877707.8 | layer 13 | |
| 5124.607896 | 14.0264502 | 665462.3 | layer 14 | 5′ ladder strand + K of |
| 5430.630469 | 14.8805006 | 823222.6 | layer 14 | RNA-A (16 nt-19 nt) (1st |
| 5775.667219 | 15.7327353 | 403978.7 | layer 14 | strand of this layer: 17 nt 5′ |
| 6104.739612 | 17.358985 | 497837.6 | layer 14 | ladder + K) |
| 941.0997742 | 1.00617129 | 434917.1 | layer 15 | Dehydrated 5′ ladder |
| 1247.123193 | 1.00617129 | 372773.9 | layer 15 | fragments of RNA-B (3 nt- |
| 1552.164982 | 1.08954221 | 616853.9 | layer 15 | 16 nt) (1st strand of this |
| 1881.217672 | 1.24184666 | 521765.8 | layer 15 | layer: 9 nt dehydrated 5′ |
| 2210.271149 | 1.62330149 | 472943.1 | layer 15 | ladder) |
| 2555.324154 | 2.0972215 | 419035.6 | layer 15 | |
| 2861.340205 | 2.79199317 | 624309.7 | layer 15 | |
| 3167.365722 | 3.56248457 | 565875.8 | layer 15 | |
| 3496.429326 | 4.95845325 | 170306.5 | layer 15 | |
| 3801.456263 | 6.04902711 | 443266.8 | layer 15 | |
| 4130.516332 | 8.06233673 | 211494.2 | layer 15 | |
| 4435.545107 | 9.14528358 | 214453.2 | layer 15 | |
| 4741.570855 | 10.2285127 | 356995.4 | layer 15 | |
| 5046.61168 | 11.312251 | 528960.1 | layer 15 | |

Chance Multiple Internal Fragments have the Same Combination.

The chance of multiple internal fragments with the same combination, thus, the same mass, can be calculated. The chance is dependent on the specific combination. Several examples are listed in Table 3, the trend shows that as the fragments get longer, the chance of having identical combination (or mass) quickly decreases. Since a ladder fragment intensity is at least 20 times stronger than an internal fragment, a few multiple mass values hits are going to pose challenges for the nesting algorithm.

TABLE 4
Occurrence of identical combinations at different length from a 100 nt RNA.

| | | | | | | Likely |
|---|---|---|---|---|---|---|
| | | | | Chance of | Number | number of |
| | | | Most | most | of | strands with |
| | Monoisotopic | Number of | common | common | internal | this |
| Length | Mass (Da) | combinations | combination | combination | strands | combination |
| 3 | 930-1050 | 20 | 1110 | 9.4% | 96 | 9 |
| 6 | 1900-2100 | 84 | 2211 | 4.4% | 93 | 4 |
| 9 | 2850-3150 | 220 | 3222 | 2.9% | 90 | 3 |
| 12 | 3800-4200 | 455 | 3333 | 2.2% | 87 | 2 |
| 15 | 4750-5250 | 816 | 4442 | 1.5% | 86 | 1 |

TABLE 5
Sample t-test results of MS intensity ratio from 3′ ladders and MS intensity
ratio from both 3′ and 5′ ladders of the synthesized 100 nt RNA.
Both p-value = 0.6356288 and p-value = 0.4201398 are greater than
the threshold of 0.05, therefore, we fail to reject the null hypothesis that
the population mean value of the MS intensity ratio is equal to 1.

| | Test | | | | 95% |
|---|---|---|---|---|---|
| | sample | | Test | | Confidence |
| Data for t-test | size | Hypotheses | Statistic | P-value | Interval |
| Intensity ratio of 70 3′ | 69 | H₀: Mean = 1 | 0.4759596 | 0.6356288 | (0.9593153, |
| ladders (3 nts-72 nts) | | H₁: Mean ≠ 1 | | | 1.0969652) |
| Intensity ratio of 70 3′ | 135 | H₀:Mean = 1 | 0.8086676 | 0.4201398 | (0.9248851, |
| ladders (3 nts-72 nts) and | | H₁:Mean ≠ 1 | | | 1.1221718) |
| 67 5′ ladders (2 nts-68 nts) | | | | | |

TABLE 6
The accuracy of ladder strand identification using
the NGMS- method. The accuracy maintained is 100%.

| | | | | | | Ratio |
|---|---|---|---|---|---|---|
| | | | | | | between |
| | | | | | | layer |
| | | | | | | strongest |
| | | | | | | Intensity and |
| | | | | Correct | Wrong | global |
| | Layer | Strongest data | Strongest | Ladder | Ladder | strongest |
| No | Identity | point | Intensity | Count | Count | ladder |
| 1 | 3′ ladder strands | 9 nt 3′ ladder of | 19565443.86 | 18 | 0 | 1 |
| | of RNA-A (2 nt- | RNA-A | | | | |
| | 19 nt) | | | | | |
| 2 | 5′ ladder strands | 14 nt 5′ ladder of | 13773512.73 | 17 | 0 | 0.703971391 |
| | of RNA-A (3 nt- | RNA-A | | | | |
| | 19 nt) | | | | | |
| 3 | 3′ ladder strands | 15 nt 3′ ladder of | 5658018.71 | 17 | 0 | 0.289184276 |
| | of RNA-B (3 nt- | RNA-B | | | | |
| | 19 nt) | | | | | |
| 4 | Dehydrated 5′ | 11 nt dehydrated | 4506335.34 | 17 | 0 | 0.23032114 |
| | ladder strands of | 5′ ladder of | | | | |
| | RNA-A (3 nt- | RNA-A | | | | |
| | 19 nt) | | | | | |
| 5 | 5′ ladder strands | 5 nt 5′ ladder of | 3707474.08 | 17 | 0 | 0.189490926 |
| | of RNA-B (3 nt- | RNA-B | | | | |
| | 19 nt) | | | | | |
| 6 | 3′ ladder strand + | 9 nt 3′ladder + | 2364349.34 | 16 | 0 | 0.120843123 |
| | K of RNA-A | K of RNA-A | | | | |
| | (4 nt-19 nt) | | | | | |
| 7 | 3′ ladder strand + | 9 nt 3′ladder + Na | 1733280.51 | 7 | 0 | 0.088588867 |
| | Na of RNA-A | of RNA-A | | | | |
| | (3 nt-9 nt) | | | | | |
| 8 | 3′ ladder strand + | 12 nt | 1616993.78 | 10 | 0 | 0.082645392 |
| | Na of RNA-A | 3′ladder + Na of | | | | |
| | (11 nt-19 nt) | RNA-A | | | | |
| 9 | 3′ ladder strands | 14 nt 3′ ladder of | 1385036.14 | 17 | 0 | 0.070789917 |
| | of RNA-C (2 nt- | RNA-C | | | | |
| | 18 nt) | | | | | |
| 10 | 5′ ladder strands | 16 nt 5′ ladder of | 1343114.62 | 8 | 0 | 0.068647286 |
| | of RNA-C (12 nt- | RNA-C | | | | |
| | 19 nt) | | | | | |
| 11 | 5′ ladder strand + | 14 nt | 1305096 | 18 | 0 | 0.066704135 |
| | Na of RNA-A | 5′ladder + Na of | | | | |
| | (2 nt-19 nt) | RNA-A | | | | |
| 12 | 5′ ladder strand + | 14 nt 5′ladder + | 1204202.16 | 12 | 0 | 0.061547398 |
| | K of RNA-A | K of RNA-A | | | | |
| | (3 nt-14 nt) | | | | | |
| 13 | 5′ ladder strands | 10 nt 5′ ladder of | 877707.81 | 8 | 0 | 0.044860102 |
| | of RNA-C (3 nt- | RNA-C | | | | |
| | 10 nt) | | | | | |
| 14 | 5′ ladder strand + | 17 nt 5′ladder + K | 823222.56 | 4 | 0 | 0.042075333 |
| | K of RNA-A | of RNA-A | | | | |
| | (16 nt-19 nt) | | | | | |
| 15 | Dehydrated 5′ | 9 nt dehydrated 5′ | 624309.68 | 14 | 0 | 0.031908792 |
| | ladder strands of | ladder of RNA-B | | | | |
| | RNA-B (3 nt- | | | | | |
| | 16 nt) | | | | | |
| 16 | 3′ ladder strand + | 9 nt 3′ | 566558.8 | 9 | 1 | 0.028957115 |
| | K + Na of RNA- | ladder + K + Na of | | | | |
| | A (5 nt-11 nt) | RNA-A | | | | |
| 17 | 3′ ladder strand + | 15 nt 3′ ladder + | 548751.43 | 3 | 0 | 0.028046971 |
| | K of RNA-B | K of RNA-B | | | | |
| | (13 nt-15 nt) | | | | | |
| 18 | 3′ ladder strand + | 15 nt 3′ | 522517.84 | 13 | 1 | 0.026706158 |
| | Na of RNA-B | ladder + Na of | | | | |
| | (6-8 nt and 10- | RNA-B | | | | |
| | 19 nt) | | | | | |
| 19 | 5′ ladder strand + | 10 nt 5′ ladder + | 405849.82 | 8 | 0 | 0.020743195 |
| | K of RNA-B | K of RNA-B | | | | |
| | (10 nt-17 nt) | | | | | |
| 20 | Possible Internal | A possible 5 nt | 367230.31 | 0 | 4 | 0.018769332 |
| | | internal fragment | | | | |
| 21 | | A possible 4 nt | 334021.96 | | | 0.017072036 |
| | | internal fragment | | | | |

| | | | |
|---|---|---|---|
| Sum | 233 | 6 | 239 |

TABLE 7
Experimental rₐᵥ calculated based on the hydrolysis
sample of three 20 nt synthetic RNAs (original intensity
ratio: RNA-A:RNA-B:RNA-C = 9:3.008:0.604).

| | Experimental rₐᵥ |
|---|---|
| Geometric mean | 0.010775671 |
| Arithmetic mean | 0.012351897 |

TABLE 8
Mass difference between parallel sequences

| Δ Mass (Da) | Cause | Explanation |
|---|---|---|
| −18.01 | Dehydration | −2H −O |
| −16.00 | Gto A | −O |
| +0.99 | A to I, or C to U | −H −N +O |
| +2.01 | Hydrogenation | +2H |
| +14.01 | Methylation | +C +2H |
| +15.97 | U to s⁴U | −O +S |
| +16.00 | A to G | +O |
| +21.98 | Na adduct | −H +Na |
| +37.95 | K adduct | −H +K |
| +42.01 | Acetylation | +2C +2H +O |
| +55.96 | K adduct + hydration | +H +K +O |

TABLE 9
Base calling mismatch probability analysis at 2 mass
values using level 1 and level 2 search library.

| | Reference | Mis- | | |
|---|---|---|---|---|
| | mass | identification | Combination | Probability |
| 5158.676 | 5464.701 | U | 4445 | 0.012480 |
| | 5464.739 | U | 9215 | 0.000237 |
| | 5487.691 | A | 0674 | 0.000237 |
| | 5487.729 | A | 5444 | 0.012480 |
| | 5487.766 | A | (10)214 | 0.000118 |
| | 5503.724 | G | 4454 | 0.012480 |
| | 5503.761 | G | 9224 | 0.000594 |
| | | Total Level 1 | | 0.0386 |
| | 5466.707 | D | 9017 | 0.000011 |
| | 5477.744 | mC | 3752 | 0.002852 |
| | 5477.782 | mC | 8522 | 0.001069 |
| | 5478.728 | mU | 3653 | 0.006656 |
| | 5478.766 | mU | 8423 | 0.001782 |
| | 5501.756 | mA | 4652 | 0.004992 |
| | 5501.793 | mA | 9422 | 0.000594 |
| | 5517.750 | mG | 3662 | 0.003328 |
| | 5517.788 | mG | 8432 | 0.001782 |
| | | Total level 2 | | 0.0617 |
| 2565.316 | 2870.357 | C | 1323 | 0.019226 |
| | 2871.341 | U | 1224 | 0.014419 |
| | 2910.361 | G | 1233 | 0.019226 |
| | | Total Level 1 | | 0.053 |
| | 2873.309 | D | 1026 | 0.000961 |
| | 2884.384 | mC | 0531 | 0.001922 |
| | 2885.368 | mU | 0432 | 0.004806 |
| | 2908.395 | mA | 1431 | 0.009613 |
| | 2924.390 | mG | 0441 | 0.002403 |
| | | Total level 2 | | 0.073 |

TABLE 10
Base calling mismatch probability analysis with
selected intensity ratio and rₐᵥ = 0.02.

| Intensity | | Length | s = intensity | Relative | p | Proba- |
|---|---|---|---|---|---|---|
| ratio | rₐᵥ | k | ratio | error of rₐᵥ | value | bility |
| 1:1 | any | any | 1 | any | 0 | 0.5 |
| 10:9 | 0.02 | 20 | 1.11 | 0.2 | 0.61 | 0.27 |
| 5:4 | 0.02 | 20 | 1.25 | 0.2 | 1.3 | 0.10 |
| 4:3 | 0.02 | 20 | 1.33 | 0.2 | 1.6 | 0.051 |
| 3:2 | 0.02 | 20 | 1.5 | 0.2 | 2.3 | 0.012 |
| 5:3 | 0.02 | 20 | 1.67 | 0.2 | 2.8 | 0.0025 |
| 2:1 | 0.02 | 20 | 2 | 0.2 | 3.7 | 0.00013 |
| 7:3 | 0.02 | 20 | 2.33 | 0.2 | 4.3 | 0.000008 |
| 3:2 | 0.02 | 10 | 1.5 | 0.2 | 1.7 | 0.045 |
| 3:2 | 0.02 | 15 | 1.5 | 0.2 | 1.9 | 0.026 |
| 3:2 | 0.02 | 25 | 1.5 | 0.2 | 2.7 | 0.0033 |
| 3:2 | 0.02 | 30 | 1.5 | 0.2 | 3.4 | 0.00034 |
| 3:2 | 0.02 | 35 | 1.5 | 0.2 | 4.2 | 0.000002 |

TABLE 11
Mole fraction of sequences read from Nested Algorithm

| | Relative | | |
|---|---|---|---|
| # of layer read | intensity to the | | |
| from nested | most abundant | Standard | |
| algorithm | (sgRNA) | deviation | Notes |
| 3^{rd} layer | 23 | 6 | Unrelated to sgRNA |
| 4^{th} layer | 15 | 6 | Unrelated to sgRNA |
| 5^{th} layer | 22 | E | #6-16 agree with sgRNA |
| | | | dehydro 5′- sequence |
| 6^{th} layer | 17 | 6 | Agree with sgRNA |
| | | | 3′-+Na |
| 10^{th} layer | 8 | 6 | Unrelated to sgRNA |
| 11^{th} layer | 13 | 5 | Unrelated to sgRNA |
| 12^{th} layer | 12 | 2 | #6-17 agree with sgRNA |
| | | | 5′-+Na |
| 15^{th} layer | 10 | 5 | Unrelated to sgRNA |
| 16^{th} layer | 8 | 6 | Unrelated to sgRNA |
| 19^{th} layer | 7 | 3 | Unrelated to sgRNA |
| 29^{th} layer | 6 | 3 | Unrelated to sgRNA |
| 30^{th} layer | 6 | 4 | Agree with sgRNA |
| | | | 5′-+57 Da |
| 31^{st} layer | 5 | 3 | Agree with sgRNA |
| | | | 3′-+57 Da |
| 41^{st} layer | 17 | 10 | Agree with sgRNA |
| | | | 5′-+Et₃N |

What is claimed:

1. A method for generating the sequence of one or more RNA molecules and detecting the presence, identity, location, and quantity of RNA nucleotide modifications on said one or more RNA molecules, allowing sequencing of each RNA and modification in a given RNA sample, said method RNA comprising (i) controlled fragmentation of the RNA sample, (ii) LC-MS measurement of RNAs intact before and after acid hydrolysis to collect data for MS sequencing and analysis, and (iii) data processing for de novo sequencing of RNA and their modifications through use of a layer-by-layer nested sequencing algorithm.

2. A method for detecting and further sequencing of impurities that co-existing with the target sequence in a RNA sample, allowing sequencing of each RNA and modification in a given RNA sample, said method RNA comprising (i) controlled fragmentation of the RNA sample, (ii) LC-MS measurement of RNAs intact before and after acid hydrolysis to collect data for MS sequencing and analysis, and (iii) data processing for de novo sequencing of RNA and their modifications through use of a layer-by-layer nested sequencing algorithm.

3. The method of claim 2, wherein the relative abundance of the impurities are provided.

4. The method of claim 2, wherein the algorithm segregates data points of consecutive ladder fragments from the same parent RNA based on the original RNA abundance order into a 3D-mass-intensity-retention time layer.

5. The method of claim 2 wherein within each layer, a short RNA sequence is read by base-calling each nucleotide from mass differences between consecutive ladder fragments thereby efficiently handling MS data separation and separate different RNA sequences on silica, eliminating the need for complex physical sample separation steps.

6. A nested algorithm comprising the steps of initiating the 1^{st} layer by identifying the data point (data point A) with a mass lower than the parent RNA and which has the highest signal intensity (i.e., the most intense potential ladder fragment) and data point A is likely associated with a ladder fragment from the most abundant RNA in the sample; (ii) starting from point A, base calling which identifies the subsequent data point (data point B) with an added mass equivalent to one nucleotide wherein point B likely corresponds to a consecutive ladder fragment from the same RNA and recording as an additional nucleotide in the RNA sequence; once data point B is identified, continuation of the process iteratively to determine subsequent data points of RNA ladder fragments.

7. The nested algorithm of claim 6, wherein searching for the next data point follows one of more of the following criteria: 1) alignment of mass differences with the searching library, encompassing four canonical nucleotides and RNA modifications; 2) fitting appropriate retention time (t_{R}) differences into a 2D t_{R} versus mass sigmoidal curve; 3) selecting the match with highest intensity in cases of multiple matches; and 4) ensuring intensities within the same order of magnitude for consecutive ladder fragments.

8. The method of claim 1 for use in verifying the target sequence of a therapeutic RNA.

9. The method of claim 1, for use in determining the relative abundance and sequences of impurities within a sample of a therapeutic RNA.

10. The method of claim 1, further comprising the step of utilizing a sequencing scoring system.

11. The method of claim 1, wherein the controlled fragmentation of the RNA is achieved by chemical degradation, enzymatic degradation, or physical degradation.

12. The method of claim 1, wherein the controlled fragmentation of the RNA is achieved by hydrolysis.

13. The method of claim 1, wherein the acid hydrolysis is achieved through use of formic acid.

14. The method of claim 1, wherein the mass measurement is achieved by LC-MS, gas chromatography, capillary electrophoresis, ion mobility spectrometry, or other methods coupled with mass spectrometry.

15. The method of claim 1, wherein the mass measurement is achieved by LC-MS.

16. A kit for use in generating the sequence of one or more RNA molecules and detecting the presence, identity, location, and quantity of RNA nucleotide modifications on said one or more RNA molecules, said kit comprising one or more components for performance of the method of claim 1.

17. The kit of claim 16, further comprising the step of utilizing a sequencing scoring system.

18. The kit of claim 16, wherein the controlled fragmentation of the RNA is achieved by hydrolysis.

19. The kit of claim 16, wherein the acid hydrolysis is achieved through use of formic acid.

20. An MS based sequencing instrument for use in generating the sequence of one or more RNA molecules and detecting the presence, identity, location, and quantity of RNA nucleotide modifications on said one or more RNA molecules, said instrument comprising one or more components for performance the method of claim 1.

21. The method of claim 1, wherein said method is computer implemented.


SEQUENCE LISTING (35 sequences)

SEQ ID NO: 1 - RNA, 18 nt, Synthetic construct
  source 1..18: mol_type = other RNA
aagcgttcadgdggcata

SEQ ID NO: 2 - RNA, 20 nt, Synthetic construct
  source 1..20: mol_type = other RNA
aaaccgttaccattactgag

SEQ ID NO: 3 - RNA, 20 nt, Synthetic construct
  source 1..20: mol_type = other RNA
tattcaagttacactcaaga

SEQ ID NO: 4 - RNA, 18 nt, Synthetic construct
  source 1..18: mol_type = other RNA
gtacatcttcccctttat

SEQ ID NO: 5 - RNA, 19 nt, Synthetic construct
  source 1..19: mol_type = other RNA
aaaccgttaccattactga

SEQ ID NO: 6 - RNA, 20 nt, Synthetic construct
  source 1..20: mol_type = other RNA
gagtcattaccattgccaaa

SEQ ID NO: 7 - RNA, 19 nt, Synthetic construct
  source 1..19: mol_type = other RNA
tattcaagttacactcaag

SEQ ID NO: 8 - RNA, 20 nt, Synthetic construct
  source 1..20: mol_type = other RNA
agaactcacattgaacttat

SEQ ID NO: 9 - RNA, 19 nt, Synthetic construct
  source 1..19: mol_type = other RNA
gcgtacatcttccccttta

SEQ ID NO: 10 - RNA, 20 nt, Synthetic construct
  source 1..20: mol_type = other RNA
tatttccccttctacatgcg

SEQ ID NO: 11 - RNA, 27 nt, Synthetic construct
  source 1..27: mol_type = other RNA
agtmcggamtggccgagtggtcgtttt

SEQ ID NO: 12 - RNA, 27 nt, Synthetic construct
  source 1..27: mol_type = other RNA
agagctagaaatagcaagttaaaataa

SEQ ID NO: 13 - RNA, 35 nt, Synthetic construct
  source 1..35: mol_type = other RNA
ggctagtccgttatcaacttgaaaaagtgcttmtt

SEQ ID NO: 14 - RNA, 23 nt, Synthetic construct
  source 1..23: mol_type = other RNA
aagctaaaagtatcgaagttctg

SEQ ID NO: 15 - RNA, 26 nt, Synthetic construct
  source 1..26: mol_type = other RNA
ctgaaaaaagtcttcgctagctaaaa

SEQ ID NO: 16 - RNA, 13 nt, Synthetic construct
  source 1..13: mol_type = other RNA
cacgatggccgag

SEQ ID NO: 17 - RNA, 30 nt, Synthetic construct
  source 1..30: mol_type = other RNA
tcgtggctgagccacggtgaaaaagttcga

SEQ ID NO: 18 - RNA, 30 nt, Synthetic construct; identical to SEQ ID NO: 17
  source 1..30: mol_type = other RNA

SEQ ID NO: 19 - RNA, 16 nt, Synthetic construct
  source 1..16: mol_type = other RNA
tttacaactctattgt

SEQ ID NO: 20 - RNA, 14 nt, Synthetic construct
  source 1..14: mol_type = other RNA
gcgatggccgagtg

SEQ ID NO: 21 - RNA, 19 nt, Synthetic construct
  source 1..19: mol_type = other RNA
actcatgttgccatgagaa

SEQ ID NO: 22 - RNA, 13 nt, Synthetic construct
  source 1..13: mol_type = other RNA
tacgtcatgcgac

SEQ ID NO: 23 - RNA, 16 nt, Synthetic construct
  source 1..16: mol_type = other RNA
gtgacgtgtcgtggat

SEQ ID NO: 24 - RNA, 16 nt, Synthetic construct
  source 1..16: mol_type = other RNA
ttcagctatagatcct

SEQ ID NO: 25 - RNA, 15 nt, Synthetic construct
  source 1..15: mol_type = other RNA
ggccgagtggtcgtt

SEQ ID NO: 26 - RNA, 16 nt, Synthetic construct
  source 1..16: mol_type = other RNA
tggtcgttttagagct

SEQ ID NO: 27 - RNA, 19 nt, Synthetic construct
  source 1..19: mol_type = other RNA
gagtcattaccattgccaa

SEQ ID NO: 28 - RNA, 20 nt, Synthetic construct
  source 1..20: mol_type = other RNA
agaactcacattgaacttat

SEQ ID NO: 29 - RNA, 20 nt, Synthetic construct
  source 1..20: mol_type = other RNA
tatttccccttctacatgcg

SEQ ID NO: 30 - RNA, 31 nt, Synthetic construct
  source 1..31: mol_type = other RNA
gtcaggatggccgagtggtcgttttagagct

SEQ ID NO: 31 - RNA, 32 nt, Synthetic construct
  source 1..32: mol_type = other RNA
agaaatagcaagtttaaaataaggctagtccg

SEQ ID NO: 32 - RNA, 39 nt, Synthetic construct
  source 1..39: mol_type = other RNA
ttattcaacttgaaaaagtggcaccgagtcggtgctttt

SEQ ID NO: 33 - RNA, 31 nt, Synthetic construct; identical to SEQ ID NO: 30
  source 1..31: mol_type = other RNA

SEQ ID NO: 34 - RNA, 19 nt, Synthetic construct
  source 1..19: mol_type = other RNA
agaaatagcaagtttaaaa

SEQ ID NO: 35 - RNA, 18 nt, synthetic construct
  source 1..18: mol_type = other RNA
cgttgaatcgcadgtgaa